SEQUENCE LISTING (33 sequences)

SEQ ID NO: 1 - DNA, 44 bp, Artificial Sequence
  ALS-MCS-F primer
cacatctagacacaccatggcacacttaagcacctgcacatccg

SEQ ID NO: 2 - DNA, 43 bp, Artificial Sequence
  ALS-MCS-R primer
tgtgaagcttcaattgtgtgccggatgtgcaggtgcttaagtg

SEQ ID NO: 3 - DNA, 31 bp, Artificial Sequence
  P-At-ALS-F1 primer
cgacatttacgtgtctagattatgtatttcc

SEQ ID NO: 4 - DNA, 30 bp, Artificial Sequence
  P-At-ALS-R1 primer
cgaagaagatgttgttgttgttgttgttgc

SEQ ID NO: 5 - DNA, 30 bp, Artificial Sequence
  At-ALS-F1 primer
tatctcttgctcatctttctcctgaaccat

SEQ ID NO: 6 - DNA, 30 bp, Artificial Sequence
  At-ALS-R1 primer
aggttctgataatcaccggtttcatctctc

SEQ ID NO: 7 - DNA, 30 bp, Artificial Sequence
  T-At-ALS-F1 primer
ctgagagatgaaaccggtgattatcagaac

SEQ ID NO: 8 - DNA, 33 bp, Artificial Sequence
  T-At-ALS-R1 primer
ccatgatcgagttagttactaacaattgtcaac

SEQ ID NO: 9 - DNA, 2535 bp, Arabidopsis thaliana
  promoter (1)..(2496): ALS1 gene promoter
cgacatttacgtgtctagattatgtatttccaactttcattaacaatataatcgcatataaatgaaaaatcgtttccaggataatattttgatgaaatctcatattattgttcgtactcggattgatgttgaaggcttgaagcgcttcaaattatagaccagattatttaagtttttcttttgtttactccatatcaatttgatccattatactacctaagaaaatttaggtaacatagaattatttattgttatagtaaaaaaaaggaaaaccacaaaaataatctacttttacgtatatactattttcatgacataagtaattaagttgtacaacttttttttaatgaaaagagagagtaaatttatcatgttcatgtgtagttacctcgtgaataaccgacggttatatagacgcctaacatgaattgttcagttgaagacagttcaaaacatgtgtttcactctaaaatcctcaacaaaaaaaaagtgttaaaatttgtaaacctctttcaagcaaaaaaagaaaaagtgttagaatcccaagattctttcataatccggaatcttggctgaaaacgtataaaagagattgacgtagtaacaaggagtcttggtatgcttccatgctttttatccttttttgtcatggaaccatgatttggttaccatttattatgtaaccgaaattttcattgtaataatgaatatttaaatttttagcaaaaaaaaacaaaaaaaaacaaggagtcttgtcttcgttctcaaatttcagagctcttgcacttttcaagagttttactttgatgagtgagacatttgtctttttagtgtttattttctaaacttaaaatagtagcatcaacatcactcaattataattcttaagatgttgtagaaaaatattttatagatggaaagtaatcgatattaagacaaataagaaaccaaaccggactttgtgttcagaccgaatcaaatctgaattggagaaattatggtggaggcgaaagtcaacggaactaaagtataaaaccaaatgtcaaaaataaaacccaattttcatccttaaacgaacctgctgaaaccctaatttcgattaccaattccgatctaaaaagaagtcatggaagccattgattccgcaatcgatcctctcagagatttcgctaagagcagtgttcgtctcgtccagcgctgtcacaaacccgatcgcaagggtaacgccttttctcaaaaaaatctcatttccgatttttgatctgtagattagggttttctgaaattttgatatcatttgtaattgaattggttatcagaattcacgaaagtagctgtgcgtacggcgattggatttgtggtgatgggattcgttggattcttcgtgaagctcgttttcatcccaatcaacaacatcatcgttggatcttcttagtgtagtactttctttacgaggtaattgatctcgcattatatatctacattttggttatgttacttgacatatagtcattgattcaatagttctgttaattcctttaaagatcattttgactagaccacattcttggttcattcctcaataatttgtaatcatattggtggatatagaagtagattggttatagatcagatagtggaagactttaggatgaatttcagctagtttttttttttggcttattgtctcaaaagattagtgctttgctgtctccattgcttctgctatcgacacgcttctgtctccttgtatctttattatatctattcgtcccatgagttttgtttgttctgtattcgttcgctctggtgtcatggatggagtctctgttccatgtttctgtaatgcatgttgggttgtttcatgcaagaaatgctgagataaacactcatttgtgaaagtttctaaactctgaatcgcgctacaggcaatgctccgaggagtaggaggagaagaacgaaccaaacgacattatcagccctttgaggaagctcttagttttgttattgtttttgtagccaaattctccattcttattccattttcacttatctcttgttccttatagaccttataagttttttattcatgtatacaaattatattgtcatcaagaagtatctttaaaatctaaatctcaaatcaccaggactatgtttttgtccaattcgtggaaccaacttgcagcttgtatccattctcttaaccaataaaaaaagaaagaaagatcaatttgataaatttctcagccacaaattctacatttaggttttagcatatcgaaggctcaatcacaaatacaatagatagactagagattccagcgtcacgtgagttttatctataaataaaggaccaaaaatcaaatcccgagggcattttcgtaatccaacataaaacccttaaacttcaagtctcatttttaaacaaatcatgttcacaagtctcttcttcttctctgtttctctatctcttgctcatctttctcctgaaccatggcggcggcaacaacaacaacaacaacatcttcttcg

SEQ ID NO: 10 - DNA, 2434 bp, Arabidopsis thaliana
  terminator (1)..(2434): ALS1
ctgagagatgaaaccggtgattatcagaaccttttatggtctttgtatgcatatggtaaaaaaacttagtttgcaatttcctgtttgttttggtaatttgagtttcttttagttgttgatctgcctgctttttggtttacgtcagactactactgctgttgttgtttggtttcctttctttcattttataaataaataatccggttcggtttactccttgtgactggctcagtttggttattgcgaaatgcgaatggtaaattgagtaattgaaattcgttattagggttctaagctgttttaacagtcactgggttaatatctctcgaatcttgcatggaaaatgctcttaccattggtttttaattgaaatgtgctcatatgggccgtggtttccaaattaaataaaactacgatgtcatcgagaagtaaaatcaactgtgtccacattatcagttttgtgtatacgatgaaatagggtaattcaaaatctagcttgatatgccttttggttcattttaaccttctgtaaacattttttcagattttgaacaagtaaatccaaaaaaaaaaaaaaaaaaatctcaactcaacactaaattattttaatgtataaaagatgcttaaaacatttggcttaaaagaaagaagctaaaaacatagagaactcttgtaaattgaagtatgaaaatatactgaattgggtattatatgaatttttctgatttaggattcacatgatccaaaaaggaaatccagaagcactaatcagacattggaagtaggaatatttcaaaaagttttttttttttaagtaagtgacaaaagcttttaaaaaatagaaaagaaactagtattaaagttgtaaatttaataaacaaaagaaattttttatattttttcatttctttttccagcatgaggttatgatggcaggatgtggatttcatttttttccttttgatagccttttaattgatctattataattgacgaaaaaatattagttaattatagatatattttaggtagtattagcaatttacacttccaaaagactatgtaagttgtaaatatgatgcgttgatctcttcatcattcaatggttagtcaaaaaaataaaagcttaactagtaaactaaagtagtcaaaaattgtactttagtttaaaatattacatgaataatccaaaacgacatttatgtgaaacaaaaacaatatctagaaaattacatgctcttttttcttcttcttttttgacaaaaaaaaacatctttaattccgatatattgtaaactatatgctgtttttgttttcatcatttcattctttttgttttcatcaatggatttcattgatatgttaaacatgagttacacaacagaccacgtaacaccaataaagattgagacttcttcggaaactaattaaagtttagttgtagatggtgttttatcgtttatatgcatgttagaaatttctaatgaggatgacgtgtattagattaattaaaaaaaacgtatacatgatcggtggtacgtaactatatgagtctatatgggaaactggaatttagttataacgatataccgtatacaacaaattaaatcgagcttgttctttgttttgttaaacaacaaattaaattgaactatttatattttgttgatatttttttcaagtgaagaaagtgcaaattgcataaagctaattttaatcgaaactacatatactcataatctatacgaagaaactgaatgcagttgatatagaaaagtagatccaaccaaataacaataaaaaataaatacttatgtaactttcaacgttatattaattattatatacttacatgtaaatcaggatcaacctttattttccatagcacgttcataaaaaaaaaatatcgaacatagagaaaccactcatattaattactgttgcttatataaattggtcaatcttcaaatgtgtaattggtgagttgttcaaacttcaaagagtgagaccctttcgcctttctcagacattaaaagtaaagactctcactttctctcctttttacttttggcccatttatatacaaaataaaatctcttgacttggcatttttttttttcactttttctttcgtcaatgagtaaaatattttaaatagttatagtatatgttacatatatttctgtctcttgggccatctgtatagtactgaatatactattattgccctcaaaattatacacgaacatcaatgaaattgtaattccaaaagagtttttttttttgcgtgcaactattggaatatttgctttgtaattttatggatccgagactctttttagggtaatcgagttttcaggctctaattaaattgtttcgttcgaatatgggaatgtccaacgttgacaattgttagtaactaactcgatcatgt

SEQ ID NO: 11 - DNA, 819 bp, Cauliflower mosaic virus
  promoter (276)..(819)
  promoter (276)..(815)
gaattcgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttagcttcatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcttgaccatgg

SEQ ID NO: 12 - DNA, 79 bp, Artificial Sequence
  Pr′3-F primer
cacaggtctcaccggattcctgggacgattgtactgatacctctgtcgatatgttgaaattcaggcctgaagctaaagg

SEQ ID NO: 13 - DNA, 74 bp, Artificial Sequence
  Pr3′-R primer
tgtgggtctcaccggtgctgacagactaactcgataaaccaaaaacaagcccacacctttagcttcaggcctga

SEQ ID NO: 14 - DNA, 148 bp, Papaver rhoeas
  3′UTR (16)..(118): fragment
cacaggtctcaccggcttcctgggacgattgtactgatacctctgtgcatatgttgaaattcaggcctgaagctaaaggtgtgggcttgtttttggtttatcgagttagtctgtcagcaccggtggtacccacagccactctgggtgt

SEQ ID NO: 15 - DNA, 24 bp, Artificial Sequence
  M-13 F universal primer
cgccagggttttcccagtcacgac

SEQ ID NO: 16 - DNA, 31 bp, Artificial Sequence
  ALS-A122T-R primer
gaatctccattgatgtacctccagggtaagc

SEQ ID NO: 17 - DNA, 31 bp, Artificial Sequence
  ALS-A122T-F primer
gcttaccctggaggtacatcaatggagattc

SEQ ID NO: 18 - DNA, 22 bp, Artificial Sequence
  ALS -MluI-R primer
tcgtaatcgaacgcgttacctc

SEQ ID NO: 19 - DNA, 81 bp, Artificial Sequence
  ALS-P197S-R primer
tcgaacgcgttacctcaacaatcggagtctcttgaaacgcatctgtaccaatcatacgacgagagacttgtcctgtgattg

SEQ ID NO: 20 - DNA, 28 bp, Artificial Sequence
  ALS-NheI-F primer
gctttataatgaatgtgcaagagctagc

SEQ ID NO: 21 - DNA, 37 bp, Artificial Sequence
  ALS-W574L-R primer
gctttgtagaaccgatcttccaattgcataaccatgc

SEQ ID NO: 22 - DNA, 37 bp, Artificial Sequence
  ALS-W574L-F primer
gcatggttatgcaattggaagatcggttctacaaagc

SEQ ID NO: 23 - DNA, 24 bp, Artificial Sequence
  M-13 R reverse primer
agcggataacaatttcacacagga

SEQ ID NO: 24 - DNA, 2069 bp, Arabidopsis thaliana
  exon (29)..(2038): ALS1 CDS
tatctcttgctcatctttctcctgaaccatggcggcggcaacaacaacaacaacaacatcttcttcgatctccttctccaccaaaccatctccttcctcctccaaatcaccattaccaatctccagattctccctcccattctccctaaaccccaacaaatcatcctcctcctcccgccgccgcggtatcaaatccagctctccctcctccatctccgccgtgctcaacacaaccaccaatgtcacaaccactccctctccaaccaaacctaccaaacccgaaacattcatctcccgattcgctccagatcaaccccgcaaaggcgctgatatcctcgtcgaagctttagaacgtcaaggcgtagaaaccgtattcgcttaccctggaggtgcatcaatggagattcaccaagccttaacccgctcttcctcaatccgtaacgtccttcctcgtcacgaacaaggaggtgtattcgcagcagaaggatacgctcgatcctcaggtaaaccaggtatctgtatagccacttcaggtcccggagctacaaatctcgttagcggattagccgatgcgttgttagatagtgttcctcttgtagcaatcacaggacaagtccctcgtcgtatgattggtacagatgcgtttcaagagactccgattgttgaggtaacgcgttcgattacgaagcataactatcttgtgatggatgttgaagatatccctaggattattgaggaagctttctttttagctacttctggtagacctggacctgttttggttgatgttcctaaagatattcaacaacagcttgcgattcctaattgggaacaggctatgagattacctggttatatgtctaggatgcctaaacctccggaagattctcatttggagcagattgttaggttgatttctgagtctaagaagcctgtgttgtatgttggtggtggttgtttgaattctagcgatgaattgggtaggtttgttgagcttacggggatccctgttgcgagtacgttgatggggctgggatcttatccttgtgatgatgagttgtcgttacatatgcttggaatgcatgggactgtgtatgcaaattacgctgtggagcatagtgatttgttgttggcgtttggggtaaggtttgatgatcgtgtcacgggtaagcttgaggcttttgctagtagggctaagattgttcatattgatattgactcggctgagattgggaagaataagactcctcatgtgtctgtgtgtggtgatgttaagctggctttgcaagggatgaataaggttcttgagaaccgagcggaggagcttaagcttgattttggagtttggaggaatgagttgaacgtacagaaacagaagtttccgttgagctttaagacgtttggggaagctattcctccacagtatgcgattaaggtccttgatgagttgactgatggaaaagccataataagtactggtgtcgggcaacatcaaatgtgggcggcgcagttctacaattacaagaaaccaaggcagtggctatcatcaggaggccttggagctatgggatttggacttcctgctgcgattggagcgtctgttgctaaccctgatgcgatagttgtggatattgacggagatggaagctttataatgaatgtgcaagagctagccactattcgtgtagagaatcttccagtgaaggtacttttattaaacaaccagcatcttggcatggttatgcaatgggaagatcggttctacaaagctaaccgagctcacacatttctcggggatccggctcaggaggacgagatattcccgaacatgttgctgtttgcagcagcttgcgggattccagcggcgagggtgacaaagaaagcagatctccgagaagctattcagacaatgctggatacaccaggaccttacctgttggatgtgatttgtccgcaccaagaacatgtgttgccgatgatcccgagtggtggcactttcaacgatgtcataacggaaggagatggccggattaaatactgagagatgaaaccggtgattatcagaacct

SEQ ID NO: 25 - DNA, 1061 bp, Arabidopsis thaliana
  exon (63)..(156): SEC-61 gene exon 1
atccttaaacgaacctgctgaaaccctaatttcgattaccaattccgatctaaaaagaagtcatggaagccattgattccgcaatcgatcctctcagagatttcgctaagagcagtgttcgtctcgtccagcgctgtcacaaacccgatcgcaagggtaacgccttttctcaaaaaaatctcatttccgatttttgatctgtagattagggttttctgaaattttgatatcatttgtaattgaattggttatcagaattcacgaaagtagctgtgcgtacggcgattggatttgtggtgatgggattcgttggattcttcgtgaagctcgttttcatcccaatcaacaacatcatcgttggatcttcttagtgtagtactttctttacgaggtaattgatctcgcattatatatctacattttggttatgttacttgacatatagtcattgattcaatagttctgttaattcctttaaagatcattttgactagaccacattcttggttcattcctcaataatttgtaatcatattggtggatatagaagtagattggttatagatcagatagtggaagactttaggatgaatttcagctagtttttttttttggcttattgtctcaaaagattagtgctttgctgtctccattgcttctgctatcgacacgcttctgtctccttgtatctttattatatctattcgtcccatgagttttgtttgttctgtattcgttcgctctggtgtcatggatggagtctctgttccatgtttctgtaatgcatgttgggttgtttcatgcaagaaatgctgagataaacactcatttgtgaaagtttctaaactctgaatcgcgctacaggcaatgctccgaggagtaggaggagaagaacgaaccaaacgacattatcagccctttgaggaagctcttagttttgttattgtttttgtagccaaattctccattcttattccattttcacttatctcttgttccttatagaccttataagttttttattcatgtatacaaattatattgtcatcaagaagtatctt

SEQ ID NO: 26 - DNA, 30 bp, Artificial Sequence
  P-ALS-SacI-F primer
gagtcttgtcttcgttctcaaatttcagag

SEQ ID NO: 27 - DNA, 40 bp, Artificial Sequence
  mTATA-R primer
ctacaacatcttaagaattcgaattgagtgatgttgatgc

SEQ ID NO: 28 - DNA, 40 bp, Artificial Sequence
  mTATA-F primer
gcatcaacatcactcaattcgaattcttaagatgttgtag

SEQ ID NO: 29 - DNA, 45 bp, Artificial Sequence
  mTATA R primer
gggttttatttttgacatttggttttcgactttagttccgttgac

SEQ ID NO: 30 - DNA, 45 bp, Artificial Sequence
  mTATA F primer
gtcaacggaactaaagtcgaaaaccaaatgtcaaaaataaaaccc

SEQ ID NO: 31 - DNA, 22 bp, Artificial Sequence
  At-ALS-Pvu-R primer
gagaaaaggcgttacccttgcg

SEQ ID NO: 32 - DNA, 2340 bp, Arabidopsis thaliana
  exon (137)..(2146): ALS1 CDS with 3 point mutations
cctaggtccgcgacgttgtaaaacgacggccagtgaattcgattaattaaggccggccagatctgcggccgctcatgatgtacactcgagggcgcgcccccgggtctagagtgcacgagctcggcgcccggccgccatggcggcggcaacaacaacaacaacaacatcttcttcgatctccttctccaccaaaccatctccttcctcctccaaatcaccattaccaatctccagattctccctcccattctccctaaaccccaacaaatcatcctcctcctcccgccgccgcggtatcaaatccagctctccctcctccatctccgccgtgctcaacacaaccaccaatgtcacaaccactccctctccaaccaaacctaccaaacccgaaacattcatctcccgattcgctccagatcaaccccgcaaaggcgctgatatcctcgtcgaagctttagaacgtcaaggcgtagaaaccgtattcgcttaccctggaggtacatcaatggagattcaccaagccttaacccgctcttcctcaatccgtaacgtccttcctcgtcacgaacaaggaggtgtattcgcagcagaaggatacgctcgatcctcaggtaaaccaggtatctgtatagccacttcaggtcccggagctacaaatctcgttagcggattagccgatgcgttgttagatagtgttcctcttgtagcaatcacaggacaagtctctcgtcgtatgattggtacagatgcgtttcaagagactccgattgttgaggtaacgcgttcgattacgaagcataactatcttgtgatggatgttgaagatatccctaggattattgaggaagctttctttttagctacttctggtagacctggacctgttttggttgatgttcctaaagatattcaacaacagcttgcgattcctaattgggaacaggctatgagattacctggttatatgtctaggatgcctaaacctccggaagattctcatttggagcagattgttaggttgatttctgagtctaagaagcctgtgttgtatgttggtggtggttgtttgaattctagcgatgaattgggtaggtttgttgagcttacggggatccctgttgcgagtacgttgatggggctgggatcttatccttgtgatgatgagttgtcgttacatatgcttggaatgcatgggactgtgtatgcaaattacgctgtggagcatagtgatttgttgttggcgtttggggtaaggtttgatgatcgtgtcacgggtaagcttgaggcttttgctagtagggctaagattgttcatattgatattgactcggctgagattgggaagaataagactcctcatgtgtctgtgtgtggtgatgttaagctggctttgcaagggatgaataaggttcttgagaaccgagcggaggagcttaagcttgattttggagtttggaggaatgagttgaacgtacagaaacagaagtttccgttgagctttaagacgtttggggaagctattcctccacagtatgcgattaaggtccttgatgagttgactgatggaaaagccataataagtactggtgtcgggcaacatcaaatgtgggcggcgcagttctacaattacaagaaaccaaggcagtggctatcatcaggaggccttggagctatgggatttggacttcctgctgcgattggagcgtctgttgctaaccctgatgcgatagttgtggatattgacggagatggaagctttataatgaatgtgcaagagctagccactattcgtgtagagaatcttccagtgaaggtacttttattaaacaaccagcatcttggcatggttatgcaattggaagatcggttctacaaagctaaccgagctcacacatttctcggggatccggctcaggaggacgagatattcccgaacatgttgctgtttgcagcagcttgcgggattccagcggcgagggtgacaaagaaagcagatctccgagaagctattcagacaatgctggatacaccaggaccttacctgttggatgtgatttgtccgcaccaagaacatgtgttgccgatgatcccgagtggtggcactttcaacgatgtcataacggaaggagatggccggattaaatactgatgagagatgaaaccggcttcctgggacgattgtactgatacctctgtgcatatgttgaaattcaggcctgaagctaaaggtgtgggcttgtttttggtttatcgagttagtctgtcagcaccggtggtaccactagtaagcttggcgtaatcacggtcatacgtgtttcctgtgtgaaattgttatccgct

SEQ ID NO: 33 - DNA, 2486 bp, Arabidopsis thaliana
  promoter (1)..(2481)
  mutation (864)..(865): at > cg
ctagattatgtatttccaactttcattaacaatataatcgcatataaatgaaaaatcgtttccaggataatattttgatgaaatctcatattattgttcgtactcggattgatgttgaaggcttgaagcgcttcaaattatagaccagattatttaagtttttcttttgtttactccatatcaatttgatccattatactacctaagaaaatttaggtaacatagaattatttattgttatagtaaaaaaaaggaaaaccacaaaaataatctacttttacgtatatactattttcatgacataagtaattaagttgtacaacttttttttaatgaaaagagagagtaaatttatcatgttcatgtgtagttacctcgtgaataaccgacggttatatagacgcctaacatgaattgttcagttgaagacagttcaaaacatgtgtttcactctaaaatcctcaacaaaaaaaaagtgttaaaatttgtaaacctctttcaagcaaaaaaagaaaaagtgttagaatcccaagattctttcataatccggaatcttggctgaaaacgtataaaagagattgacgtagtaacaaggagtcttggtatgcttccatgctttttatccttttttgtcatggaaccatgatttggttaccatttattatgtaaccgaaattttcattgtaataatgaatatttaaatttttagcaaaaaaaaacaaaaaaaaacaaggagtcttgtcttcgttctcaaatttcagagctcttgcacttttcaagagttttactttgatgagtgagacatttgtctttttagtgtttattttctaaacttaaaatagtagcatcaacatcactcaattcgaattcttaagatgttgtagaaaaatattttatagatggaaagtaatcgatattaagacaaataagaaaccaaaccggactttgtgttcagaccgaatcaaatctgaattggagaaattatggtggaggcgaaagtcaacggaactaaagtcgaaaaccaaatgtcaaaaataaaacccaattttcatccttaaacgaacctgctgaaaccctaatttcgattaccaattccgatctaaaaagaagtcatggaagccattgattccgcaatcgatcctctcagagatttcgctaagagcagtgttcgtctcgtccagcgctgtcacaaacccgatcgcaagggtaacgccttttctcaaaaaaatctcatttccgatttttgatctgtagattagggttttctgaaattttgatatcatttgtaattgaattggttatcagaattcacgaaagtagctgtgcgtacggcgattggatttgtggtgatgggattcgttggattcttcgtgaagctcgttttcatcccaatcaacaacatcatcgttggatcttcttagtgtagtactttctttacgaggtaattgatctcgcattatatatctacattttggttatgttacttgacatatagtcattgattcaatagttctgttaattcctttaaagatcattttgactagaccacattcttggttcattcctcaataatttgtaatcatattggtggatatagaagtagattggttatagatcagatagtggaagactttaggatgaatttcagctagtttttttttttggcttattgtctcaaaagattagtgctttgctgtctccattgcttctgctatcgacacgcttctgtctccttgtatctttattatatctattcgtcccatgagttttgtttgttctgtattcgttcgctctggtgtcatggatggagtctctgttccatgtttctgtaatgcatgttgggttgtttcatgcaagaaatgctgagataaacactcatttgtgaaagtttctaaactctgaatcgcgctacaggcaatgctccgaggagtaggaggagaagaacgaaccaaacgacattatcagccctttgaggaagctcttagttttgttattgtttttgtagccaaattctccattcttattccattttcacttatctcttgttccttatagaccttataagttttttattcatgtatacaaattatattgtcatcaagaagtatctttaaaatctaaatctcaaatcaccaggactatgtttttgtccaattcgtggaaccaacttgcagcttgtatccattctcttaaccaataaaaaaagaaagaaagatcaatttgataaatttctcagccacaaattctacatttaggttttagcatatcgaaggctcaatcacaaatacaatagatagactagagattccagcgtcacgtgagttttatctataaataaaggaccaaaaatcaaatcccgagggcattttcgtaatccaacataaaacccttaaacttcaagtctcatttttaaacaaatcatgttcacaagtctcttcttcttctctgtttctctatctcttgctcatctttctcctgaaccatgg

(19) United States
(12) Patent Application Publication
(10) Pub. No.: US 2013/0117887 A1
(43) Pub. Date: May 9, 2013

(54) Herbicide resistant Camelina Sativa

(71) Applicants: Seppo Paavo Kaijalainen, Helsinki (FI); Kimmo Koivu, Itasalmi Helsinki (FI); Viktor Kuvshinov, Vantaa (FI); Eric Murphy, Grand Forks, ND (US)

(72) Inventors: Seppo Paavo Kaijalainen, Helsinki (FI); Kimmo Koivu, Itasalmi Helsinki (FI); Viktor Kuvshinov, Vantaa (FI); Eric Murphy, Grand Forks, ND (US)

(73) Assignee: Agragen Inc

(21) Appl. No.: 13/200,951

(22) Filed: Oct. 5, 2011

(30) Foreign Application Priority Data
Jun. 15, 2010 (US) PCT/US2010/001704

(51) Int. Cl. C12N 15/82

(52) U.S. Cl. CPC C12N 15/8274; C12N 15/8278
USPC 800/287; 800/300; 435/320.1

(57) ABSTRACT

This disclosure provides a novel herbicide resistant plant, a method to transform Camelina sativa for herbicide resistance and a method for an improved in vitro regeneration of transformed plants.

### FIELD OF THE INVENTION

This invention relates to herbicide resistant crop plants. More specifically this invention relates to herbicide resistant Camelina sativa plants.

### BACKGROUND OF THE INVENTION

Camelina sativa (L. Crantz) belongs to the family Brassicaceae in the tribe Sisymbrieae and both spring- and winter forms are in production. It is a low-input crop adapted to low fertility soils. Results from long-term experiments in Central Europe have shown that the seed yields of Camelina sativa are comparable to the yields of oil seed rape.

As Camelina sativa is a minor crop species, very little has been done in terms of its breeding aside from testing different accessions for agronomic traits and oil profiles. However, due to the high oil content of Camelina sativa seeds (varying between 30-40%), there has been a renewed interest in Camelina sativa oil. Camelina sativa seeds have high content of polyunsaturated fatty acids, about 50-60% with an excellent balance of useful fatty acids including 30-40% of alpha-linolenic acid, which is omega-3 oil. Omega-3 oils from plants metabolically resemble marine omega-3oils and are rarely found in other seed crops. Furthermore, Camelina sativa seeds contain high amount of tocopherols (appr. 600 ppm) with a unique oxidative stability. Moreover, there is an increasing interest in Camelina sativa as animal feed.

In addition, there is an impeding need to introduce commercial crops to provide vegetable oils for biofuel production without displacing food crops from rich soils. Because Camelina sativa is well suited to marginal soils, this plant species offers an alternative crop that can be grown and harvested in large quantities. However, because of limited breeding success, improvements in Camelina sativa, such as herbicide resistance, increased protein quality, increased oil content, and enhanced agronomic characteristics are lacking.

Many herbicides used in grain production (e.g. wheat) can carry over, resulting in loss of Camelina yields. Herbicides commonly used in the Pacific Northwest America are listed in Table 1 below. Areas where these herbicides are used cannot be used for Camelina cultivation before herbicide residues are degraded. Factors that affect degradation include climate factors such as moisture, and temperature as well as soil pH. In Pacific Northwest America the period that the soil contains herbicide residues may last several years.

TABLE 1
Herbicides commonly used in the Pacific Northwest America

| | |
|---|---|
| Sulfuronic herbicide | Clorsulfuron (Glean ®) |
| | Metsulfuron (Ally ®) |
| | Tiasulfuron (Amber ®) |
| | Prosulfuron (Peak ®) |
| | Thfensulfuron combined with tribenuron |
| | (Harmony Extra ®) |
| | Sulfosulfuron (Maverick ®) |
| Imidazolinones | Imazamox (Beyond ®) |
| Sulfonylamino-carbonyl- | Propoxycarbazone-sodium (Olympus ®) |
| triazolinones | |

This invention is aimed to resolve the existing problems: creating an herbicide resistant Camelina sativa line would allow access for Camelina cultivation in areas, where growing Camelina will offer a cropping choice in areas in which choices for rotation crops are limited. Moreover, this invention is aimed to provide herbicide resistant Camelina sativa plants and cultivars.

### SHORT DESCRIPTION OF THE FIGURES

FIG. 1 illustrates the procedure for DNA cloning.

FIG. 2 illustrates cloning of plant transformation vector for ALS constructs used in this disclosure

FIG. 3 illustrates cloning of Arabidopsis thaliana (A.t) ALS 1-gene and regions up- and down-stream from the gene.

FIG. 4 illustrates cloning of pC0300-P-At-ALS containing ALS promoter region of Arabidopsis thaliana.

FIG. 5 illustrates cloning of pC0300-PT-At-ALS construct containing promoter region (P) and transcription termination region (T) of ALS gene.

FIG. 6 illustrates cloning of pC0300-T-At-ALS without promoter region.

FIG. 7 illustrates cloning of pC0300-P35S-At-ALS plant transformation vector containing 35S promoter of Cauliflower Mosaic Virus and Transcription termination region of the ALS gene of Arabidopsis thaliana.

FIG. 8 illustrates cloning of pVK1-At-ALS containing coding region of ALS gene amplified in PCR on genomic DNA of Arabidopsis thaliana.

FIG. 9 illustrates cloning of Papaver rhoeas ALS 3′UTR downstream of A.t ALS1-gene in pVK1 cloning vector.

FIG. 10 illustrates mutagenesis of Arabidopsis thaliana ALS 1 gene.

FIG. 11 illustrates cloning of mutated ALS1 gene into plant transformation vectors.

FIG. 12 illustrates the mutation (removal) of SEC61 putative TATA-boxes

FIG. 13 In vitro leaf segment test of two transgenic C. sativa cv. Celine lines VK6-A1-No24 (shown as 24 on Petri dishes) and VK6-Al-No26 (shown as 26 on Petri dishes). As a control, leaf segments of herbicide resistant natural mutant of Camelina microcarpa (C.m.) and leaf segments of susceptible C. sativa (C.s.) were used. Leaf segments of transgenic plants are resistant to the herbicides, while non-transgenic Camelina sativa is susceptible. The media in Petri dishes contains herbicides in various concentrations from 0 mg/l in control plate to 0.001, 0.01, 0.1, 1.0 mg/l of IMI, MSU and CSU.

FIG. 14 IMI and MSU 10 day-tests applied to the shoots recovered after transformation of Blane Creek. Green and well-developed shoots are most evidently transgenic with good expression of the mutant ALS gene. Such shoots were transferred to fresh MS agar with the same herbicides to prove the resistance of the shoots.

FIG. 15. Shows a Southern Analysis blot of ten transgenic ALS-carrying plants (columns 1-10). Column 11 presents plasmid DNA carrying the gene sequence, cut with restriction enzymes and mixed with non-transgenic plant DNA as positive control (BC+M). Column 12 presents a negative control being DNA of non-transgenic Camelina sativa plant DNA mixed with 1 kb Marker (Fermentas) (BC+1 kb M). The line does not show any signal.

FIG. 16A shows seedlings of transgenic lines bearing triple (APW) mutated ALS gene: ALS 11.3 (VK10A5.3) and ALS22.3 (VK13A15.12) of the fourth generation (T3) of Camelina sativa v. Blane Creek. The seedlings were grown one week on various concentrations of herbicides MSU, CSU and IMI. The concentrations are shown in mg/l. Control plate has no herbicide in the grown media.

FIG. 16B shows seedlings of transgenic lines bearing double (PW) mutated ALS gene: ALS7.3 (KK24A14.4) ALS8.3 (KK18A8) of fourth generation (T3) of Camelina sativa v. Blane Creek. The seedlings were grown one week on the various concentrations of herbicides MSU, CSU and IMI. The concentrations are shown in mg/l. Control plate has no herbicide in the growth media.

FIG. 16C shows seedlings of transgenic lines bearing A-mutated ALS gene: A3 (VK9A3.4.1) of the second generation (T1) of Camelina sativa v. Calena, as well as A9.1 (VK14A9.1) and A9.2 (VK14A9.4.2) of the second generation (T1) Camelina sativa v. Blane Creek. The seedlings were grown one week on the various concentrations of herbicides MSU, CSU and IMI. The concentrations are shown in mg/l. Control plate has no herbicide in the grown media.

FIG. 16D shows seedlings of transgenic lines bearing P-mutated ALS gene: A4 (VK9A4.4.2) of the second generation (Ti) of Camelina sativa v. Calena and A 10 (VK14A10.16) of the second generation (T1) Camelina sativa c. Blane Creek. The seedlings were grown one week on various concentrations of herbicides MSU, CSU and IMI. The concentrations are shown in mg/l.

FIG. 16E shows seedlings of transgenic lines bearing W-mutated ALS gene: A2 (VK10A2.2) of the second generation (T1) of Camelina sativa v. Blane Creek and ALS23.3 (VK10A2.3) of the fourth generation (T3) of Camelina sativa v. Blane Creek. The seedlings were grown one week on various concentrations of herbicides MSU, CSU and IMI. The concentrations are shown in mg/l. Control plate has no herbicide in the grown media.

FIG. 16F shows seedlings of wild type control seeds of Camelina sativa c. Blane Creek. The seedlings were grown one week on the various concentrations of herbicides MSU, CSU and IMI. The concentrations are shown in mg/l. Control plate has no herbicide in the grown media.

### DESCRIPTION OF THE INVENTION

The present invention provides methods for producing novel Camelina plants and cultivars resistant to herbicides. In particular, the present invention provides novel Camelina plants, tissues and seeds that contain modified acetolactate synthase (ALS) genes and proteins that are resistant to inhibition by herbicides that normally inhibit ALS enzyme.

ALS-targeting herbicides inhibit acetolactate synthase (ALS), which is required for production of essential branched-chain amino acids such as valine, leucine, and isoleucine. The five chemical classes of ALS-inhibiting herbicides are sulfonylureas (SU), imidazolinones (IMI), triazolopyrimidines (TP), pyrimidinylthiobenzoates (PTB), and sulfonylamino-carbonyl-triazolinones (SCT). Mutations in some crop plants, for example tobacco, corn and soybean have been found conferring ALS herbicide resistance in those plants. As Camelina sativa has not been a major crop plant, such information is not available for it.

ALS—inhibiting herbicides control a wide spectrum of grass and broad leaf weeds at very low application rates. As is shown in Table 1, the commonly used herbicides in Pacific Northwest in the United States of America to control weeds are members of ALS herbicides. Accordingly, this invention provides Camelina plants resistant to ALS-inhibiting herbicides, thereby providing cultivars for large-scale production in this area.

There are various ALS mutants in the nature (e.g. ALS mutant has been documented in Arabidopsis thaliana); however, in conditions where herbicides are not present the mutant enzymes do not function as effectively as normal ALS enzymes. This fact causes cost for fitness for mutated plants in the field. This disclosure provides transgenic Camelina sativa lines that are transformed with artificial ALS enzyme variations giving resistance to IMI and SU herbicides. The transgenic plants according to this invention, do not suffer fitness cost, because they have their normal ALS enzymes functional in conditions where herbicide is not present. The additional transgenic ALS mutant works during herbicide contact and allows survival of the plant in those conditions.

This invention discloses use of Arabidopsis thaliana (A.t) ALS natural gene cassette comprising promoter, gene coding region and terminator region in creating the transgenic Camelina plants. Because Arabidopsis thaliana has only a single ALS gene per genome, it is functional in all organs and development stages. Although the ALS promoter is strong enough to confer sufficient enzymatic activity with one gene copy only, we have also chosen to use the 35S as an alternative promoter strategy because it strongly and constitutively (ubiquitously) expresses ALS in various organs. We also have used so-called TATA-less pALS (or -TATA) promoters as an alternative to natural Arabidopsis thaliana ALS promoter, because the natural promoter contains an extra small gene that encodes an additional small protein. In our—TATA constructs, this additional small gene is made non-functional by deleting TATA-box element from the promoter of extra gene.

Herbicide—ALS binding is dependent on specific 3D structures of the herbicide and of ALS protein. Generally speaking, protein structure can be changed by mutating its amino acids. Resistance to ALS-inhibiting herbicides is formed by mutated 3D structure of ALS protein and reduced binding of specific herbicide chemical to certain area of the protein. We chose to use three kinds of point mutations in the natural Arabidopsis thaliana ALS-gene in the constructs that are used to transform Camelina plants (Table 2).

TABLE 2
Three kinds of point mutations of Arabidopsis thaliana ALS gene
were selected for the constructs for transforming Camelina plants.
Mutations are named according to type and sequence number of changed
amino acid: A122 (Ala 122), P197 (Pro197) and W574 (Trp574).

| Promoter | ALS gene mutation | | Resistance |
|---|---|---|---|
| pALS | 1. | Ala 122 > Thr | IMI |
| pALS | 2. | Pro 197 > Ser | SU |
| pALS | 3. | Trp 574 > Leu | IMI + SU |
| pALS | 4. | Ala 122 > Thr and Pro 197 > Ser | IMI + SU |
| pALS | 5. | Ala 122 > Thr and Trp 574 > Leu | IMI + SU |
| pALS | 6. | Pro 197 > Ser and Trp 574 > Leu | IMI + SU |
| pALS | 7. | Ala 122 > Thr and Pro 197 > Ser | IMI + SU |
| | | and Trp 574 > Leu | |
| pALS - TATA | 1. | Trp 574 > Leu | IMI + SU |
| pALS -TATA | 2. | Ala 122 > Thr | IMI + SU |
| pALS-TATA | 3. | Pro 197 > Ser | IMI + SU |
| pALS -TATA | 4. | Ala 122 > Thr and Pro 197 > Ser | IMI + SU |
| pALS -TATA | 5. | Ala 122 > Thr and Trp 574 > Leu | IMI + SU |
| pALS -TATA | 6. | Pro 197 > Ser and Trp 574 > Leu | IMI + SU |
| pALS -TATA | 7. | Ala 122 > Thr and Pro 197 > Ser | IMI + SU |
| | | and Trp 574 > Leu | |

| | | |
|---|---|---|
| p35S | Trp 574 > Leu | IMI + SU |

In the transformation procedure we used a PC0300 plant transformation vector. We have removed all needless DNA sequences from T-DNA of the vector. Therefore, only some nucleotides from bacterial T-DNA borders are left. Moreover, no antibiotic resistance genes were used in transformation. As a result, only plant natural ALS sequences containing engineered point-mutations are transferred into the plant.

### EXAMPLE 1

### DNA Cloning

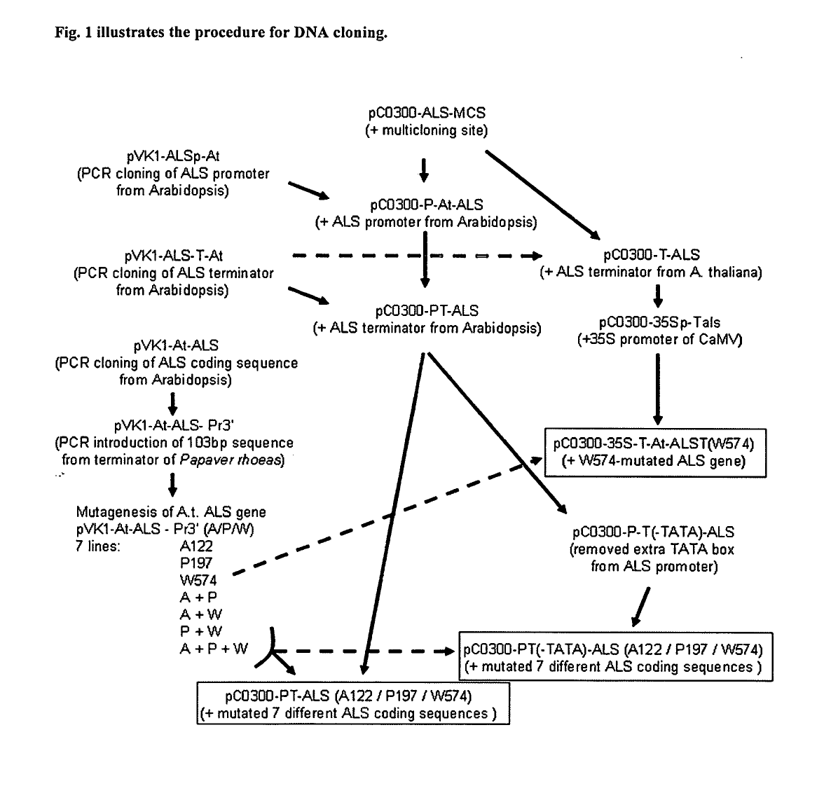

FIG. 1 illustrates the scheme of cloning plant transformation vectors containing ALS gene according to this disclosure.

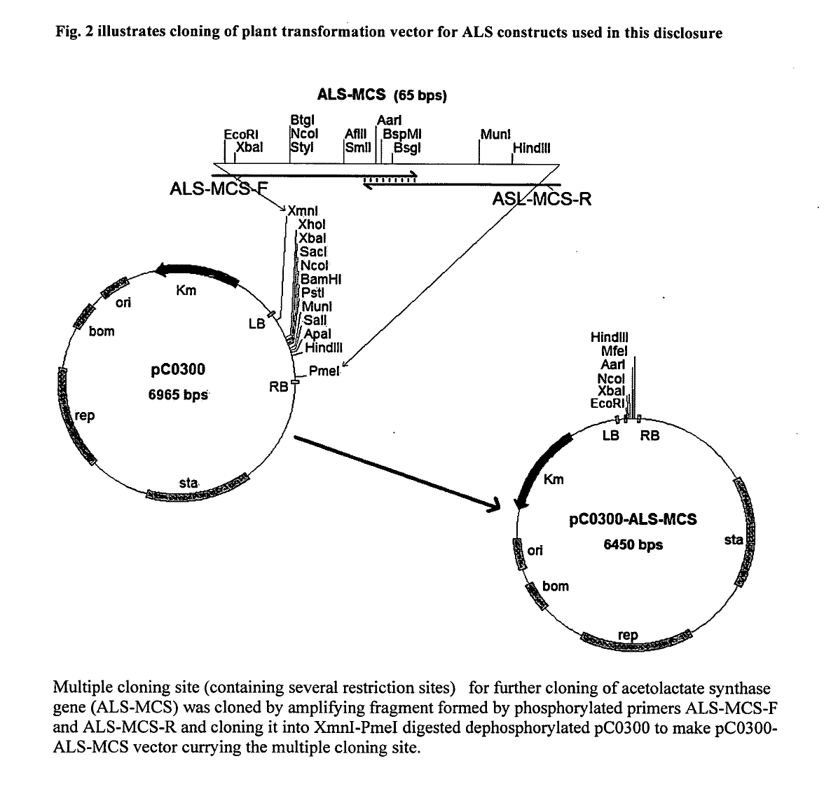

Multiple cloning sites (containing several restriction sites) for further cloning of acetolactate synthase gene (ALS-MCS) was cloned by amplifying fragment formed by phosphorylated primers ALS-MCS-F (SEQ ID NO:1) and ALS-MCS-R (SEQ ID NO:2) and cloning it into XmnI-PmeI digested dephosphorylated pC0300 to make pC0300-ALS-MCS vector carrying the multiple cloning site as is shown in FIG. 2.

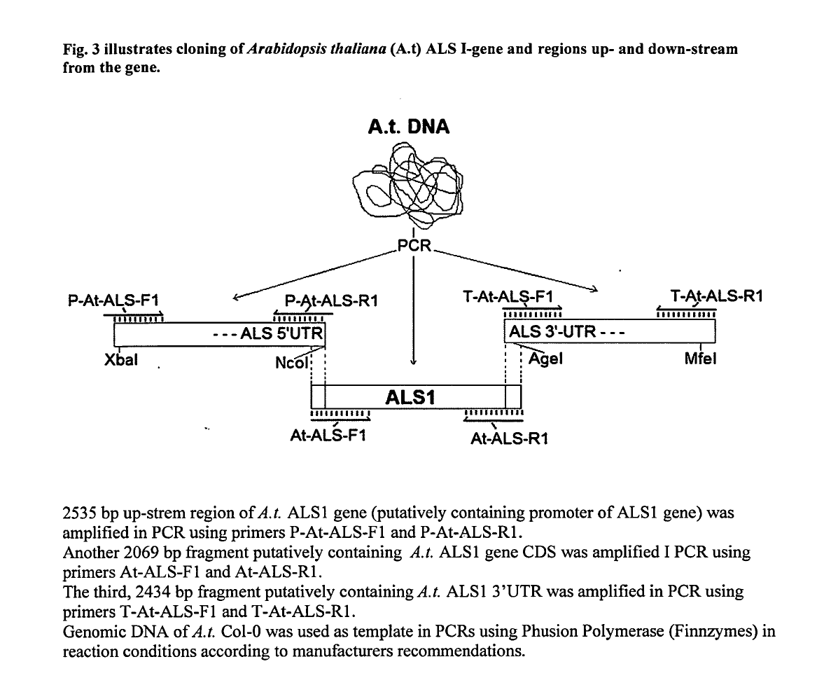

2535 by up-stream region of A.t. ALS1 gene, putatively containing promoter of ALS1 gene, was amplified in PCR using primers P-At-ALS-F1 (SEQ ID NO: 3) and P-At-ALS-R1 (SEQ ID NO: 4). Another 2069 by fragment, putatively containing A.t. ALS1 gene CDS, was amplified in PCR using primers At-ALS-F1 (SEQ ID NO: 5) and At-ALS-R1 (SEQ ID NO: 6). The third, 2434 by fragment, putatively containing A.t. ALS1 3′UTR, was amplified in PCR using primers T-At-ALS-F1 (SEQ ID NO:7) and T-At-ALS-R1 (SEQ ID NO:8). Genomic DNA of A.t. Col-0 was used as a template in PCR using Fusion Polymerase (Finnzymes) in reaction conditions according to manufacturer's recommendations. FIG. 3 illustrates cloning of Arabidopsis thaliana (A.t) ALS 1-gene and regions up-and down-stream from the gene.

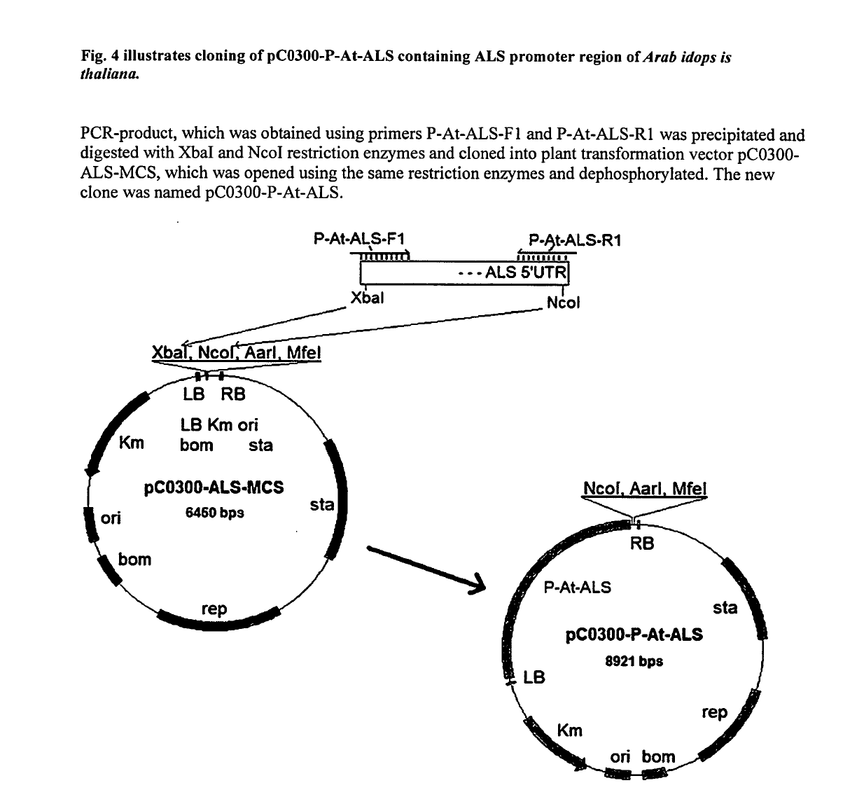

PCR-product, which was obtained by using primers P-At-ALS-F1 (SEQ ID NO: 3) and P-At-ALS-R1 (SEQ ID NO:4) was precipitated and digested with XbaI and NcoI restriction enzymes and cloned into plant transformation vector pC0300-ALS-MCS, which was opened by using the same restriction enzymes and the vector was dephosphorylated. The new clone was named as pC0300-P-At-ALS. FIG. 4 illustrates cloning of pC0300-P-At-ALS containing ALS promoter region of Arabidopsis thaliana (SEQ ID NO: 9)

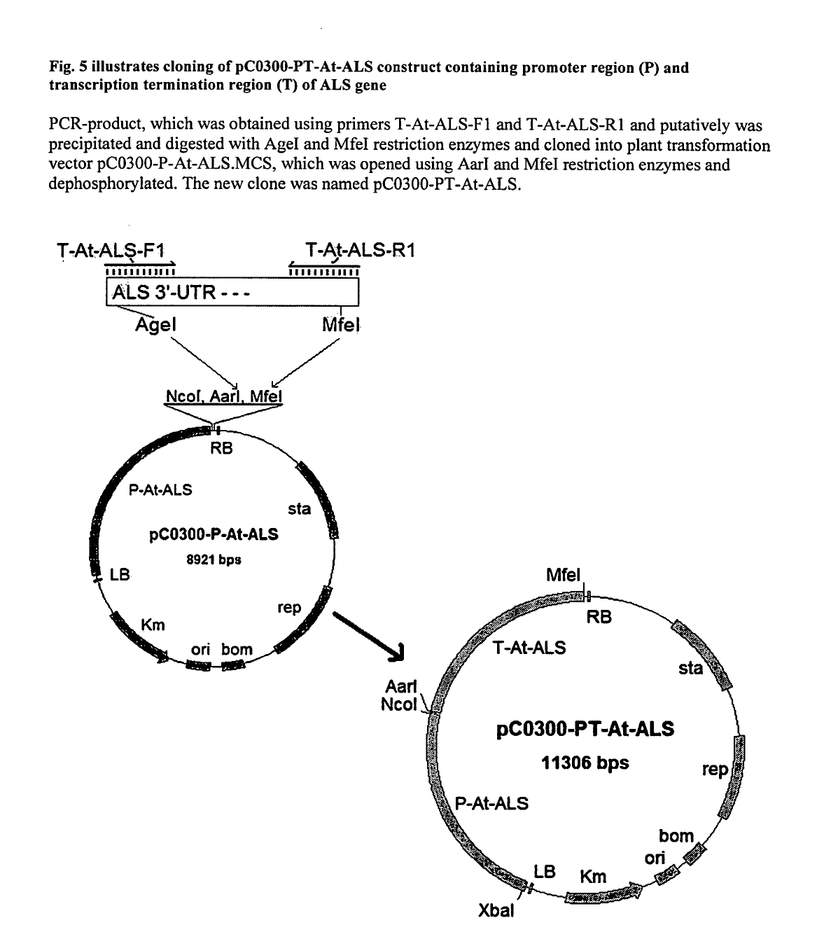

The PCR-product, was obtained by using primers T-At-ALS-F1 (SEQ ID NO:7) and T-At-ALS-R1 (SEQ ID NO:8), and putatively was precipitated and digested with AgeI and MfeI restriction enzymes, and cloned into plant transformation vector pC0300-P-At-ALS. MCS, which was opened by using AarI and MfeI restriction enzymes and dephosphorylated. The new clone was named pC0300-PT-At-ALS. FIG. 5 illustrates cloning of pC0300-PT-At-ALS construct containing promoter region (R)(SEQ ID NO:9) and transcription termination region (T) (SEQ ID NO:10) of ALS gene.

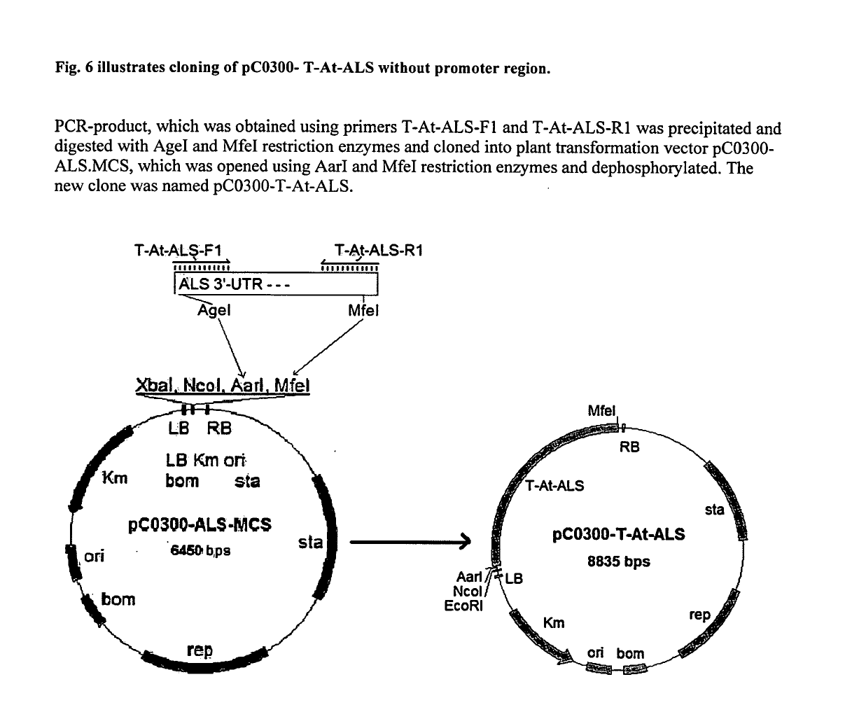

The PCR-product, which was obtained by using primers T-At-ALS-F1 (SEQ ID NO: 7) and T-At-ALS-R1 9 (SEQ ID NO:8) was precipitated and digested with AgeI and MfeI restriction enzymes, and cloned into plant transformation vector pC0300-ALS-MCS, which was opened by using AarI and MfeI restriction enzymes and dephosphorylated. The new clone was named pC0300-T-At-ALS. FIG. 6 illustrates cloning of pC0300-T-At-ALS.

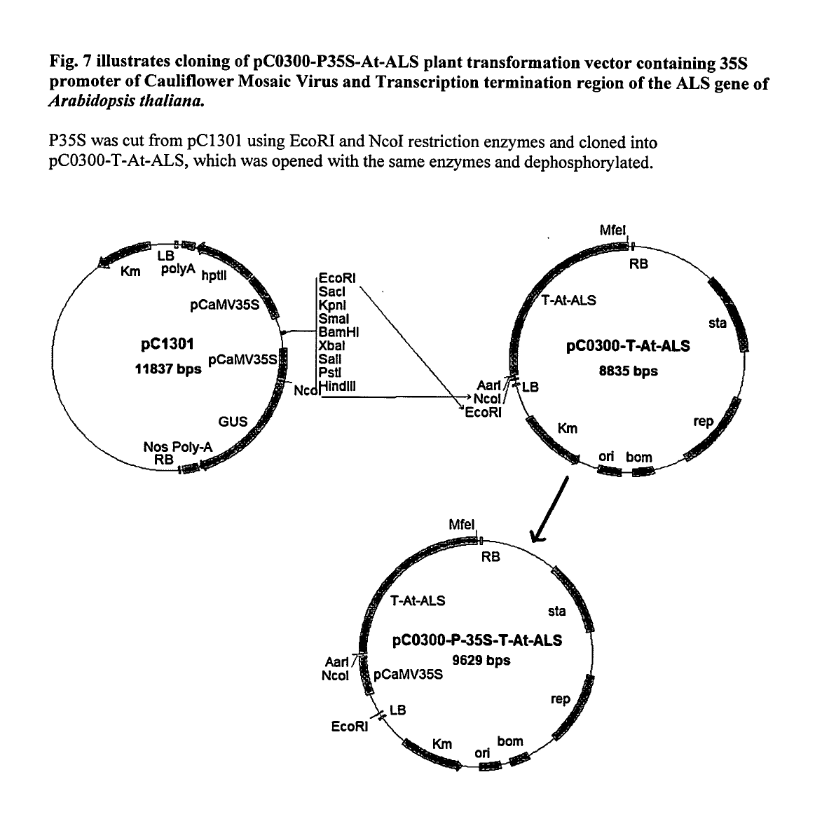

P35S was cut from pC1301 by using EcoRI and NcoI restriction enzymes and cloned into pC0300-T-At-ALS, which was opened with the same enzymes and dephosphorylated. FIG. 7 illustrates cloning of pC0300-P35S T-At-ALS plant transformation vector containing 35S promoter of Cauliflower Mosaic Virus (SEQ ID NO: 11) and Transcription termination region of the ALS gene of Arabidopsis thaliana (SEQ ID NO: 10).

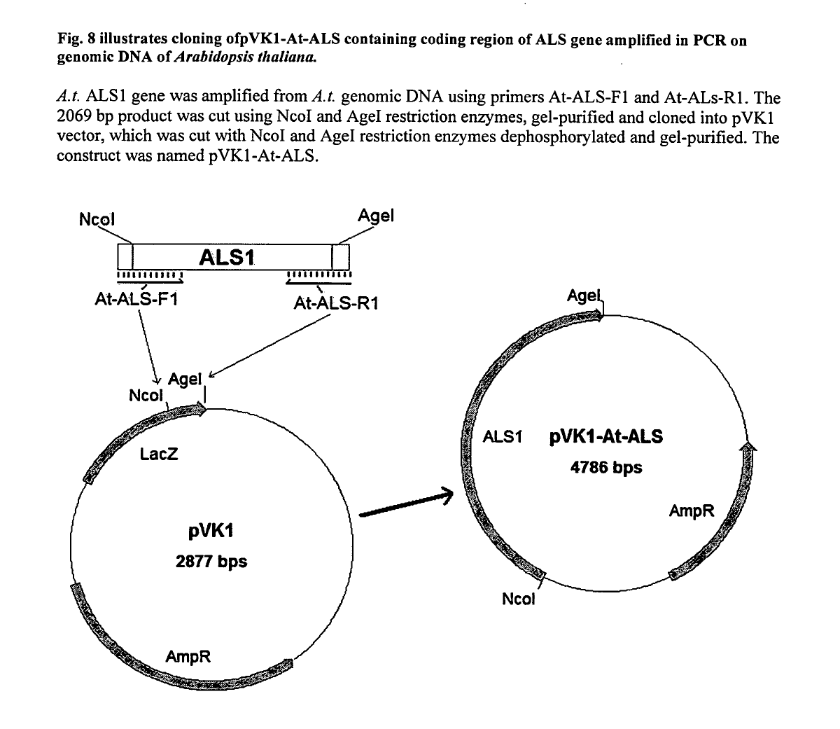

FIG. 8 illustrates cloning of pVK1-At-ALS containing coding region of ALS gene amplified in PCR on genomic DNA of Arabidopsis thaliana. A.t. ALS1 gene was amplified from A.t. genomic DNA by using primers At-ALS-F1 (SEQ ID NO: 5) and At-ALs-R1 (SEQ ID NO: 6). The 2069 by product was cut by using NcoI and AgeI restriction enzymes, gel-purified and cloned into pVK1 vector, which was cut with NcoI and AgeI restriction enzymes dephosphorylated and gel-purified. The construct was named pVK1-At-ALS.

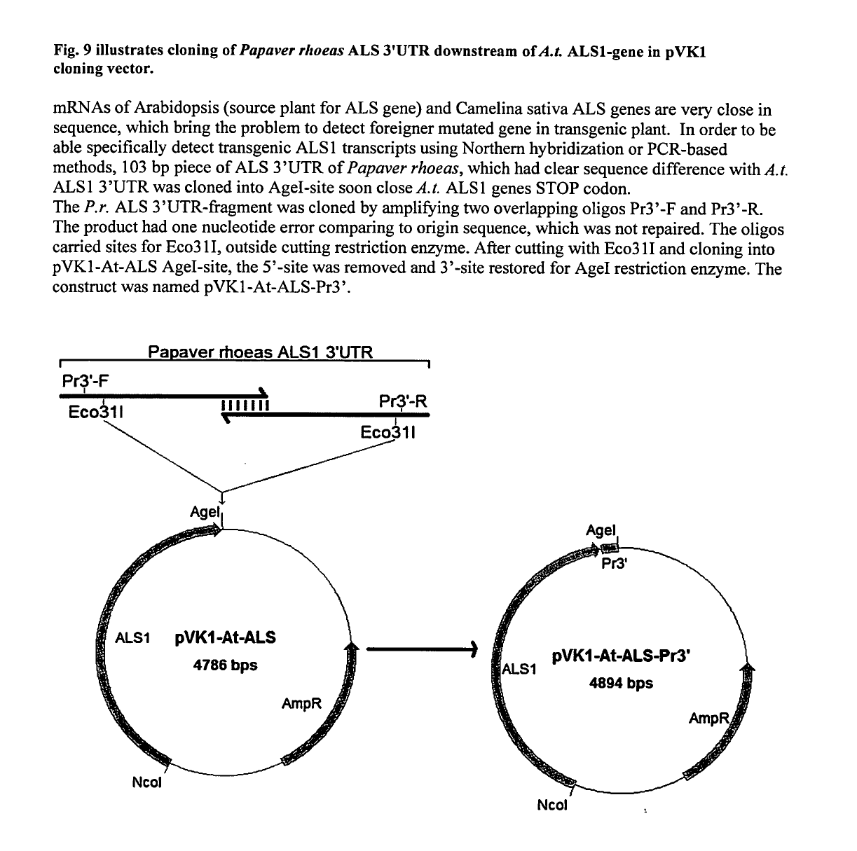

mRNA of Arabidopsis thaliana (the source plant for ALS gene) and Camelina sativa ALS genes are very close to each other by their sequence, which brings the problem of how to detect foreign mutated gene in transgenic plant. In order to be able to specifically detect transgenic ALS1 transcripts by e.g. using Northern hybridization or PCR-based methods, 103 by piece of ALS 3′UTR of Papaver rhoeas (SEQ ID NO: 14), (which had clear sequence difference with A.t. ALS1 3′UTR) was cloned into AgeI-site close after A.t ALS1 gene's STOP codon. The Papaver rhoeas ALS 3′UTR-fragment was cloned by amplifying two overlapping oligonucleotides, Pr3′-F (SEQ ID NO: 12) and Pr3′-R (SEQ ID NO: 13). The product had one nucleotide error comparing to the origin sequence, which was not repaired. The oligonucleotides carried sites for Eco31I, outside cutting restriction enzyme. After cutting with Eco31I and cloning into pVK1-At-ALS AgeI-site, the 5′-site was removed and 3′-site restored for AgeI restriction enzyme. The construct was named pVK1-At-ALS-Pr3′. FIG. 9 illustrates cloning of Papaver rhoeas ALS 3′UTR downstream of A.t. ALS 1-gene in pVK1 cloning vector.

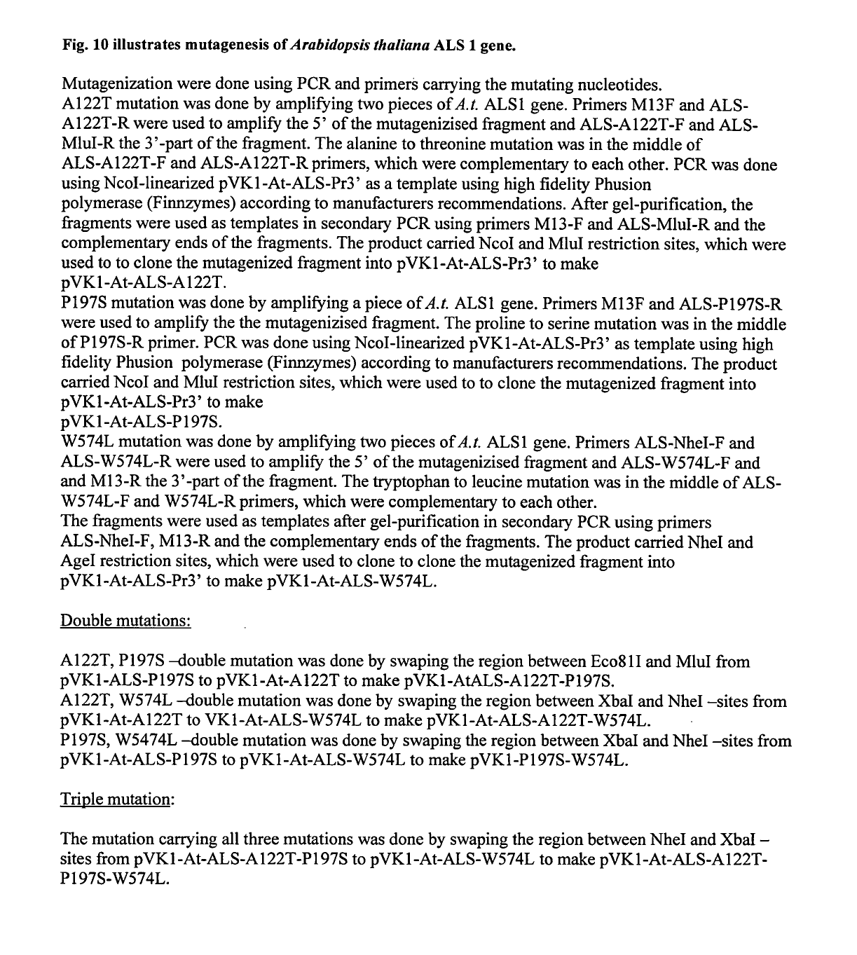

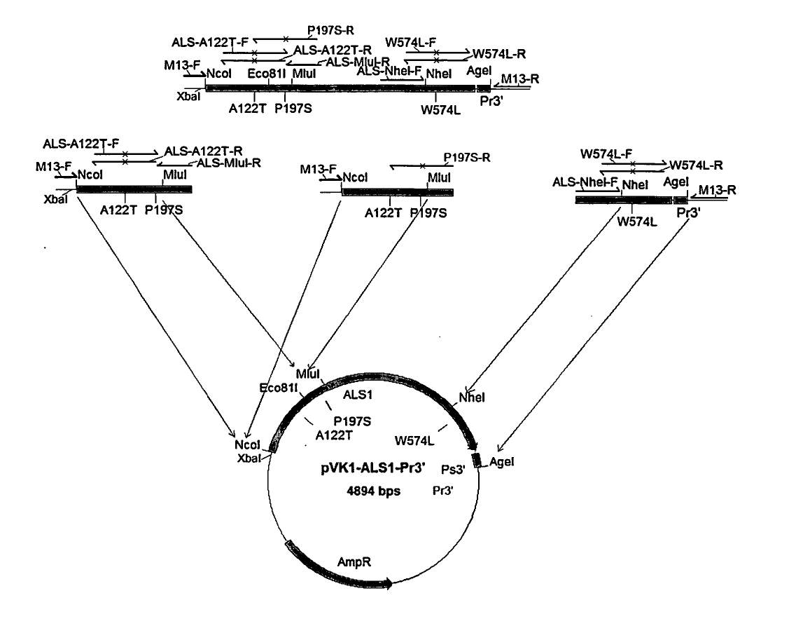

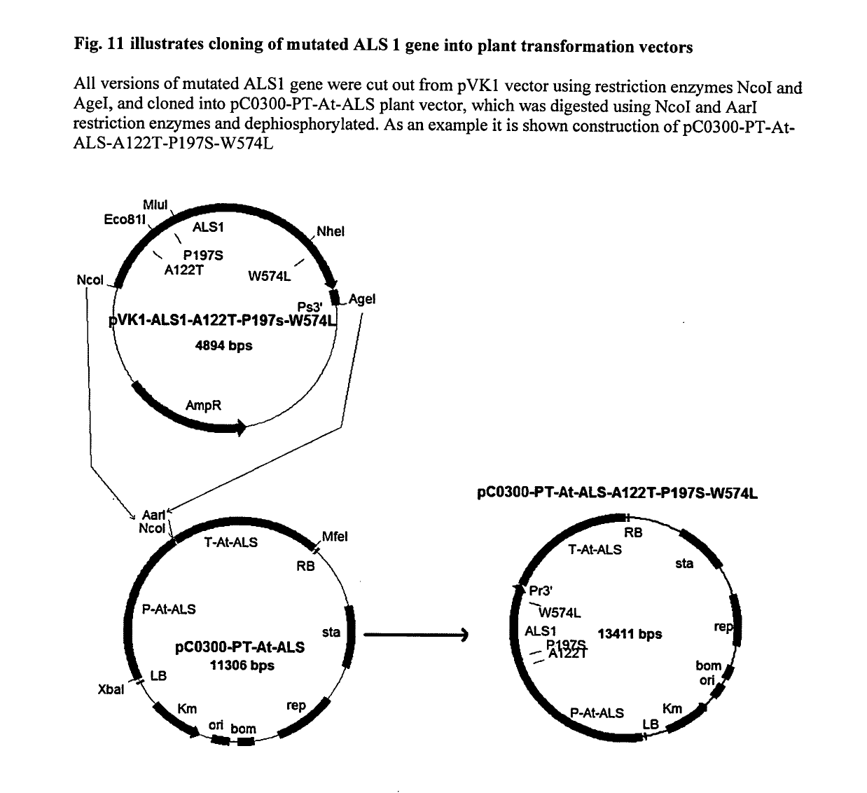

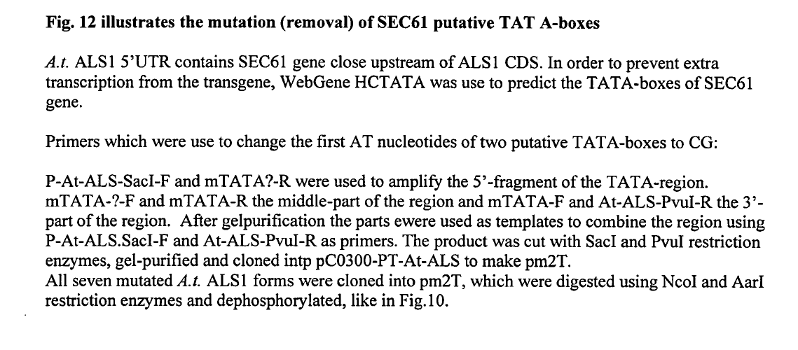

Mutagenesis was conducted by using PCR and primers carrying the mutating nucleotides. A122T mutation was conducted by amplifying two pieces of A.t. ALS1 gene. Primers M13F (SEQ ID NO: 15) and ALS-A122T-R (SEQ ID NO: 16) were used to amplify the 5′ of the mutagenized fragment, and ALS-A122T-F (SEQ ID NO: 17) and ALS-MluI-R (SEQ ID NO: 18) the 3′-part of the fragment. The alanine to threonine mutation was in the middle of ALS-A122T-F (SEQ ID NO: 17) and ALS-A122T-R (SEQ ID NO: 16) primers, which were complementary to each other. PCR was conducted by using NcoI-linearized pVK1-At-ALS-Pr3′ as a template by using high fidelity Phusion polymerase (Finnzymes) according to manufacturer's recommendations. After gel-purification, the fragments were used as templates in secondary PCR by using primers M13-F (SEQ ID NO: 15) and ALS-MluI-R (SEQ ID NO: 18), and the complementary ends of the fragments. The product carried NcoI and MluI restriction sites, which were used to clone the mutagenized fragment into pVK1-At-ALS-Pr3′ to make pVK1-At-ALS-A122T as shown in FIG. 10.

P197S mutation was created by amplifying a piece of A.t ALS1 gene. Primers M13F (SEQ ID NO: 15) and ALS-P197S-R (SEQ ID NO:19) were used to amplify the mutagenized fragment. The proline to serine mutation was in the middle of P197S-R primer. PCR was conducted by using NcoI-linearized pVK1-At-ALS-Pr3′ as template by using high fidelity Phusion polymerase (Finnzymes) according to the manufacturer's recommendations. The product carried NcoI and MluI restriction sites, which were used to clone the mutagenized fragment into pVK1-At-ALS-Pr3′ to make pVK1-At-ALS-P197S.

W574L mutation was created by amplifying two pieces of A.t ALS1 gene. Primers ALS-NheI-F (SEQ ID NO: 20) and ALS-W574L-R (SEQ ID NO: 21) were used to amplify the 5′ of the mutagenized fragment and ALS-W574L-F (SEQ ID NO:22) and M13-R (SEQ ID NO:23) the 3′-part of the fragment. The tryptophan to leucine mutation was in the middle of ALS-W574L-F (SEQ ID NO: 22) and W574L-R (SEQ ID NO: 21) primers, which were complementary to each other. The fragments were used as templates after gel-purification in secondary PCR using primers ALS-NheI-F (SEQ ID NO: 20), M13-R (SEQ ID NO: 23), and the complementary ends of the fragments. The product carried NheI and AgeI restriction sites, which were used to clone to clone the mutagenized fragment into pVK1-At-ALS-Pr3′ to make pVK1-At-ALS-W574L.

In addition to single mutations, we also created double and triple mutations in the A.t. ALS gene. A12T, P197S—double mutation was created by swapping the region between Eco81I and MluI from pVK1-ALS-P197S to pVK1-At-A122T to make pVK1-AtALS-A122T-P197S. A122T, 574L—double mutation was created by swapping the region between XbaI and NheI—sites from pVK1-At-A122T to VK1-At-ALS-W574L to make pVK1-At-ALS-A122T-W574L. P197S, W5474L—double mutation was created by swapping the region between XbaI and NheI—sites from pVK1-At-ALS-P197S to pVK1-At-ALS-W574L to make pVK1-P197S-W574L.

The mutation carrying all three mutations was created by swapping the region between NheI and XbaI—sites from pVK1-At-ALS-A122T-P197S to pVK1-At-ALS-W574L to make pVK1-At-ALS-A122T-P197S-W574L.

FIG. 11 illustrates cloning of mutated ALS1 gene into plant transformation vectors. All versions of mutated ALS1 gene were cut out from pVK1 vector by using restriction enzymes NcoI and AgeI, and cloned into pC0300-PT-At-ALS plant vector, which was digested by using NcoI and AarI restriction enzymes and dephosphorylated. As an example it is shown construction of pC0300-PT-At-ALS-A122T-P197S-W574L.

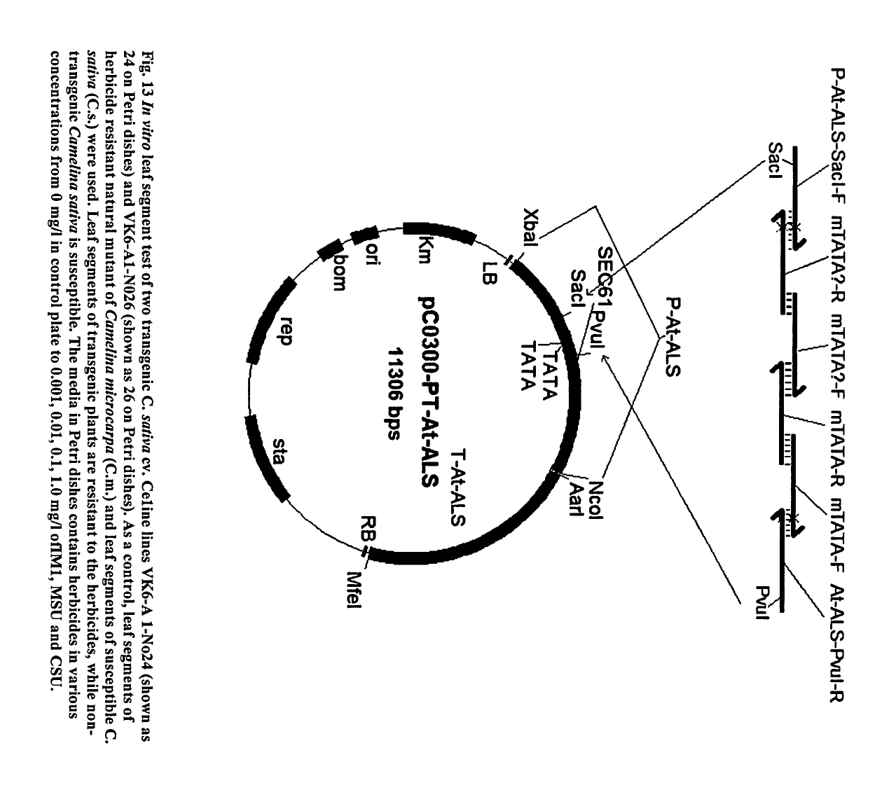

A.t. ALS1 5′UTR contains SEC61 gene (SEQ ID NO:25) close upstream of ALS1 CDS (SEQ ID NO:24). In order to prevent extra transcription from the transgene, WebGene HCTATA was used to predict the TATA-boxes of SEC61 gene. FIG. 12 illustrates the mutation (removal) of SEC61 putative TATA-boxes. Resulting mutated P-At-ALS sequence is according to SEQ ID NO:33. P-At-ALS-SacI-F (SEQ ID NO: 26) and mTATA-R (SEQ ID NO: 27) were used to amplify the 5′-fragment of the TATA-region, mTATA-F (SEQ ID NO:28) and mTATA-R (SEQ ID NO:29) the middle-part of the region and mTATA-F (SEQ ID NO:30) and At-ALS-PvuI-R (SEQ ID NO:31) the 3′-part of the region. After gel purification, the parts were used as templates to combine the region by using P-At-ALS.SacI-F (SEQ ID NO:26) and At-ALS-PvuI-R (SEQ ID NO:31) as primers. The product was cut with SacI and PvuI restriction enzymes, gel-purified and cloned into pC0300-PT-At-ALS to make pm2T. All seven mutated A.t. ALS1 forms were cloned into pm2T, which were digested by using NcoI and AarI restriction enzymes and dephosphorylated, similarly as shown in FIG. 11.

### EXAMPLE 2

### Plant Transformation

The seeds of Camelina sativa plant grown in greenhouse were sterilized by immersing in 70% ethanol for 1 min and then treating for 5 minutes in 2.5% active Cl (Na-hypoclorite) with an addition of Tween-20 (1 drop per 100 ml). After sterilization the seeds were washed three times in sterile water and placed on solid Murashige and Skoog (MS) agar medium (Murashige and Skoog, Physiol. Plant. 15:472-493, 1962) without sugars for germination. Sterilized seeds were germinated and grown 10 days on solid MS- medium without hormones. Green leaves served as a source of explants for transformation procedure.

Agrobacterium tumefaciens strain c58 carrying plasmid pC0300 containing various ALS gene mutation as described above, was grown overnight at 28° C. with shaking in liquid YEB medium (Lichtenstein and Draper, Gene Engineering of Plants. In: Glover D M (ed.) DNA Cloning—A Practical Approach, vol. 2. Oxford IRL, Oxford, pp 67-119, 1985) supplemented with 50 mg/l kanamycin and 12.5 mg/l rifampicin. Subsequently an aliquot of the culture ( 1/100 v/v) was inoculated in fresh YEB medium supplemented with 50 mg/l kanamycin and 12.5 mg/l rifampicin and the bacteria were grown overnight with shaking. Agrobacterium culture of OD.sub.600=1.0 was used in the transformation experiments.

The first true leaves of in vitro grown Camelina sativa plants cv. Calena, Celine, and Blane Creek were cut across the leaf blade. The explants were immersed for 1-3 min in MS solution inoculated with a dilution (e.g. 1/10 v/v) of the overnight culture of Agrobacterium tumefaciens. Redundant liquid on the leaf segments was removed with filter paper and the leaf segments were cultivated for 48 hours on MS 0.7% agar medium supplemented with 0.7-1.5 mg/l 6-benzylaminopurine (BAP) and 0.3-1.0 mg/l alpha-naphthaleneacetic acid (NAA). All the MS culture medias were supplemented with 2% sucrose and all in vitro cultures were kept at temperatures of 25° C. (day) and 18° C. (night) with a photoperiod of 16 h.

After the co-cultivation, the explants were washed with water containing Ticarcillin (Duchefa) 200 mg/l. The surfaces of the explants were dried on filter paper and the explants were placed on MS medium supplemented with hormones 0.7-1.5 mg/l 6-benzylaminopurine (BAP), 0.3-1.0 mg/l alpha-naphthaleneacetic acid (NAA) and 100 mg/l Ticarcillin.

After 7-10 days leaf explants were transferred to new MS medium containing 0.7-1.5 mg/l BAP 2-3% sucrose and 150-200 mg/l Ticarcillin for shoot regeneration.

Shoots developed in 7 to 14 days were cut, grown on the same media and rooted in presence of auxines. The rooted plants were transferred into soil in greenhouse.

### EXAMPLE 3

### Resistance to Herbicides and Selection of Transgenic Shoots

Because various mutations of ALS gene confer different resistance to plants, we used herbicide imidazolinon: Imazamox (IMI) and two types of sulfonylurea (SU): Metsulfuron-methyl (MSU) and Chlorsulfuron (CSU) (Sigma). It is known that mutation Al22 confers resistance to IMI, P197 to SU, and W574 to both IMI and SU.

We did not use antibiotic selection in plant transformation. We have shown before (E.G. U.S. patent application Ser. No. 12/290,379) that there is no need for selection when Camelina plants are transformed as described here and in above publication, because the transformation rate is so high. However in the case of herbicide resistance, as described here, the herbicide can also be used as selection factor.

FIG. 13 shows one of such in vitro leaf segment test of two transgenic C. sativa cv. Celine lines VK6-A1-No24 and VK6-A1-No26 transformed with Al construct (35S promoter—ALS W574 mutant—35S terminator). As controls, we used leaf segments of herbicide resistant natural ALS mutant of Camelina microcarpa that has broad resistance to class-2 herbicides and leaf segments of susceptible Camelina sativa. From the figure it can be clearly seen that leaf segments of transgenic plants are resistant to the herbicides, while non-transgenic Camelina sativa is susceptible.

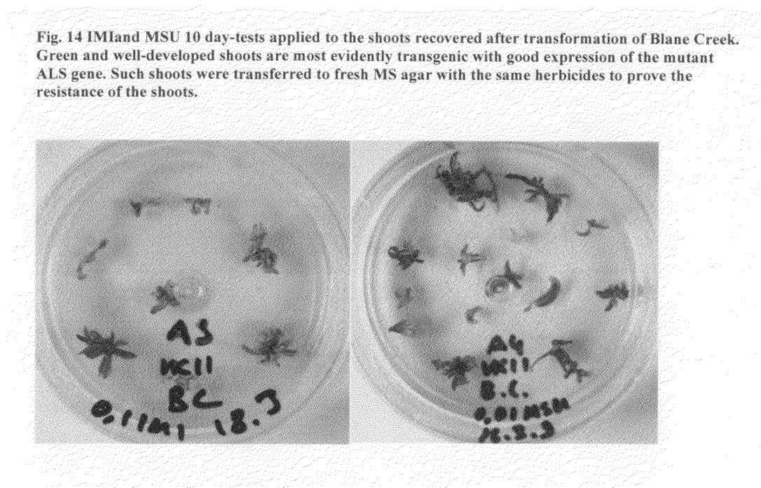

From this and other tests, it was clear that concentrations of 0.1 mg/l IMI (Al22 and W574 mutants) and 0.01 mg/l (P197 mutant) were appropriate for testing of transgenic shoots. FIG. 14 shows IMI and MSU 10 day-tests applied to the shoots recovered after transformation of Blane Creek. Green and well-developed shoots are most evidently transgenic with good expression of the mutant ALS gene.

Such shoots were transferred to fresh MS agar with the same herbicides to prove the resistance of the shoots.

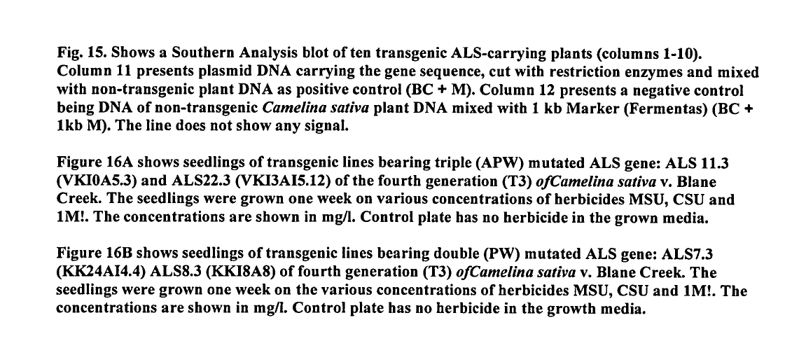

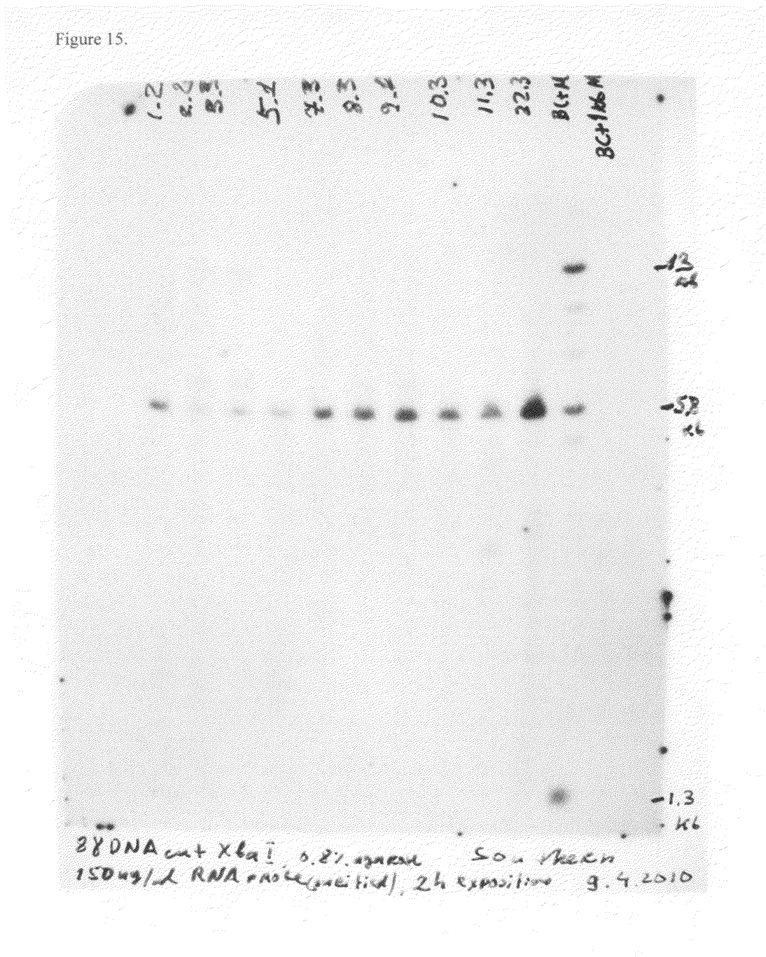

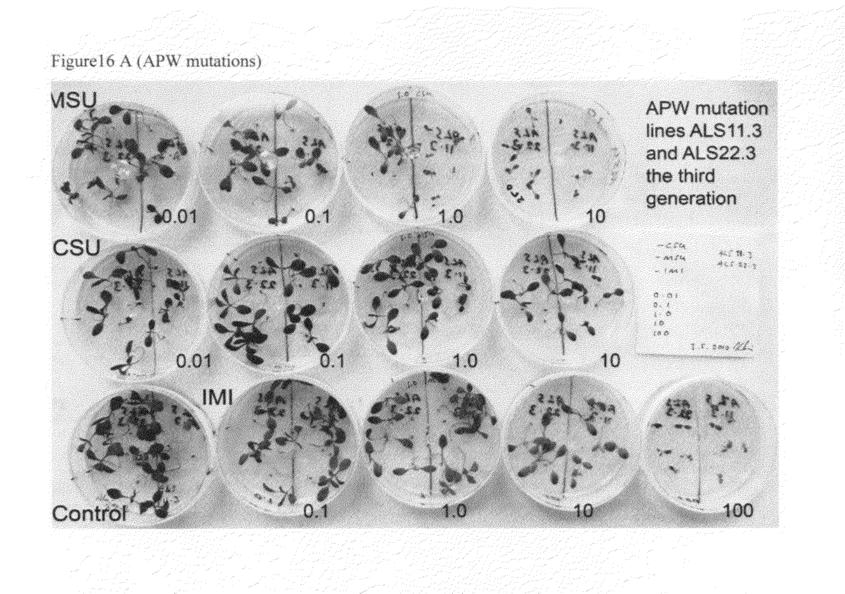

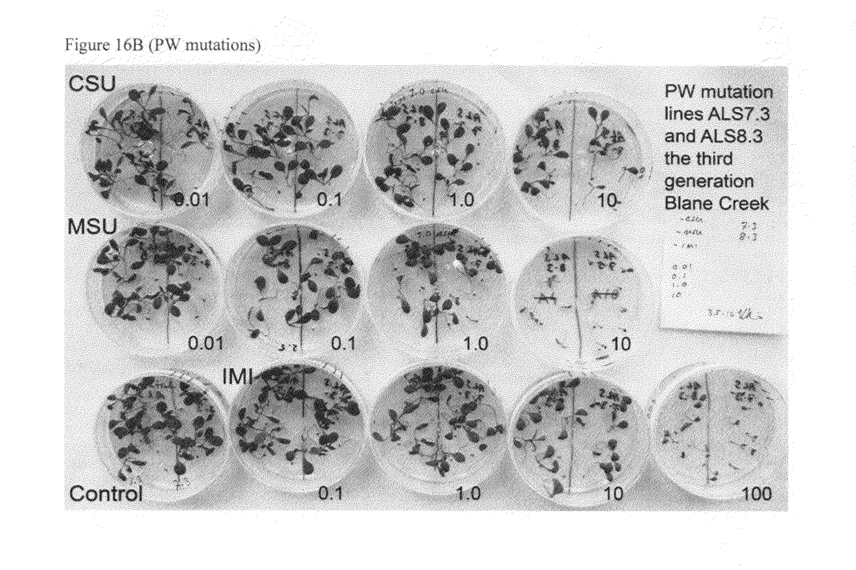

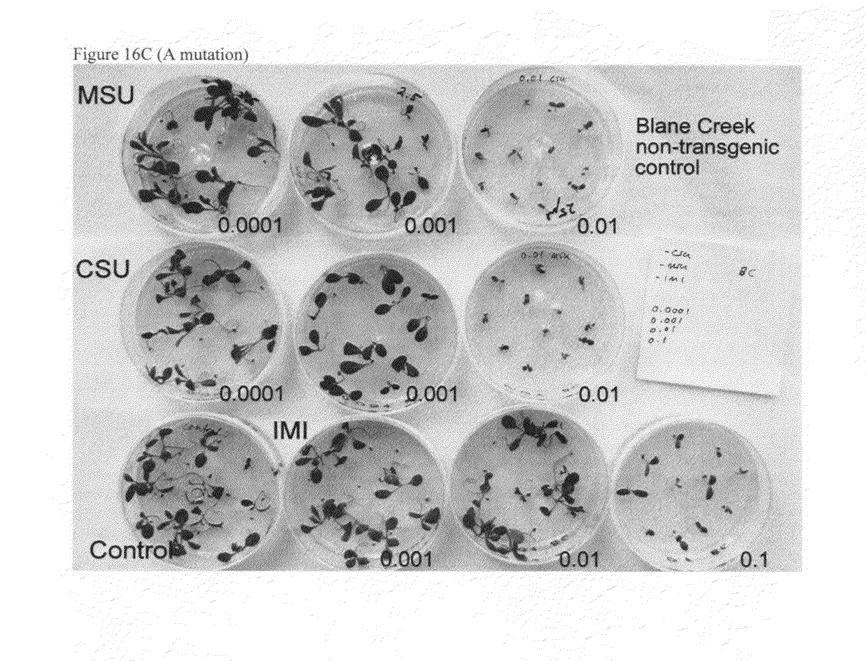

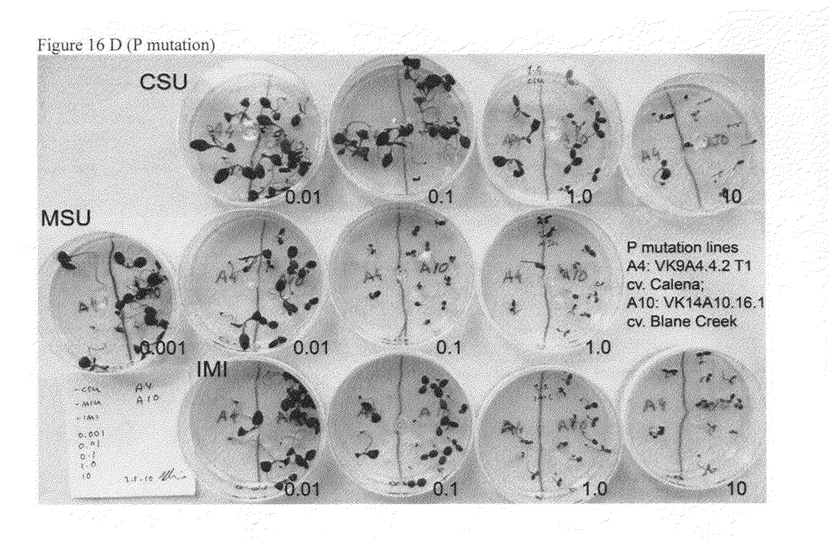

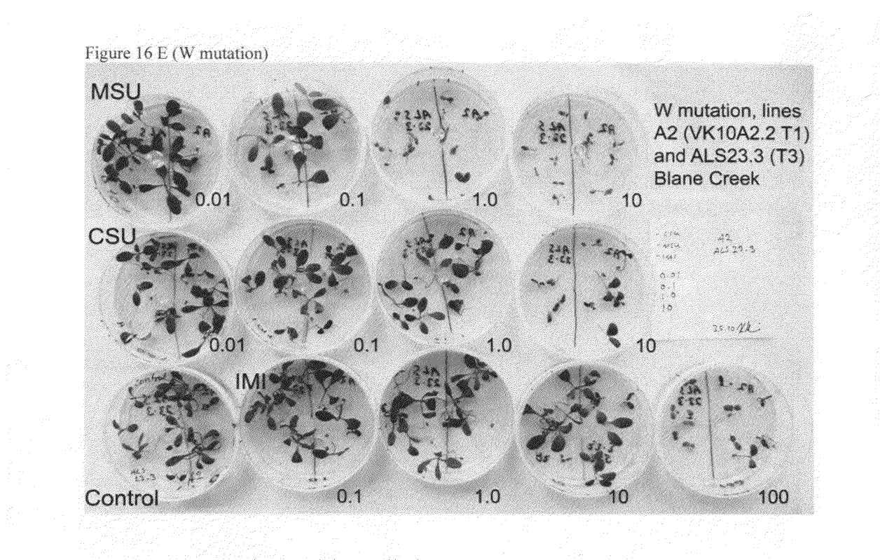

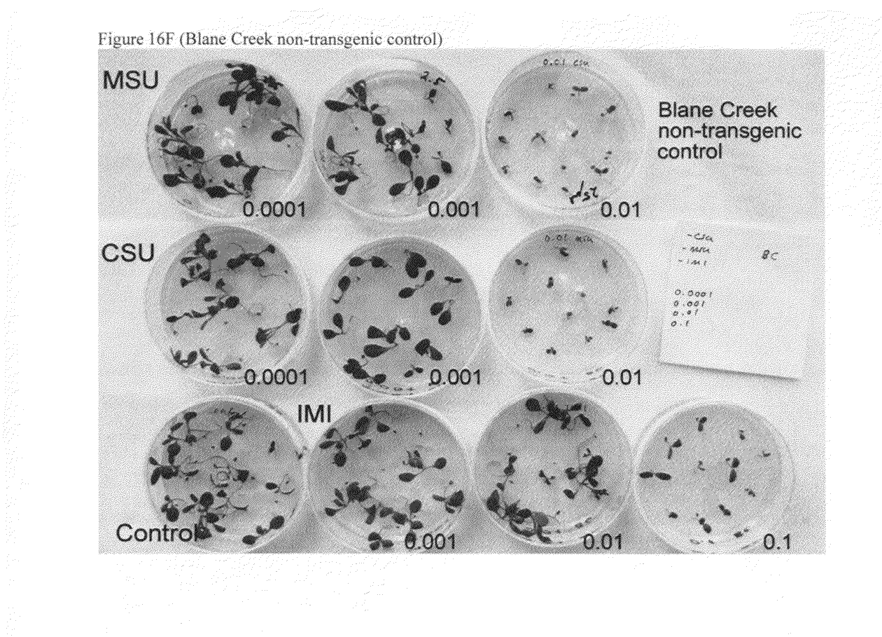

The herbicide resistance test can also be performed by using surface sterilized seeds that are germinated on the herbicide containing media similar way than leaf explants are grown. Growth of the seedlings is then followed and effects of variable herbicide concentrations are monitored, as it is shown in FIG. 16 and Table 3.

We have also developed selection of transgenic tissues with the herbicides during callus formation. Camelina sativa leaf explants 3-5 days after co-cultivation were exposed to 0.01-0.02 mg/l IMI for 3-5 days. After that the explants were returned to the same callus regeneration MS agar (0.7-1.5 mg/l BAP and 0.3-1.0 mg/l NAA). Transfer to shoot regeneration media after selection was delayed for 4-6 days. However, the majority of recovered shoots were transgenic (average 60-70% and achieving 105% transgenic shoots per transformed explants). The total number of transformed shoots also increased by 5-10 fold as compared to selection-free method.

### EXAMPLE 3

### Molecular Analysis of Transgenic Expression

PCR Analysis

Total genomic DNA was isolated from leaf tissue of transgenic and non-transgenic Camelina sativa plants by using DNeasy Plant Mini Kit according to the supplier's instructions (Qiagen). The presence of the ALS gene in the herbicide resistant plants was determined by PCR analysis by using 24 nucleotides long primers specific to the promoter sequences of ALS and hpt genes. PCR reaction mix contained approximately 1 ng/μl of template DNA and DyNAzyme polymerase (Finnzymes) was used for amplification. PCR program consisted of: 94° C. for 2 min; 30 cycles of 94° C. for 30 sec, 48° C. for 30 sec and 72° C. for 2 min. Three micro liters of PCR reaction mixture was run at 100 V in 0.8% agarose gel containing ethidium bromide. No PCR product was obtained when non-transgenic Camelina sativa DNA was used as template, whereas when using transgenic Camelina sativa an amplification product of 700 nucleotides corresponding to the positive control was obtained, which confirmed the presence of transgene in these Camelina sativa plants.

RT-PCR Assay of the RNA Expression and in the Herbicide Resistant Plants

Plant total RNA was isolated from approximately 20 mg leaf samples by using E.Z.N.A Plant RNA kit (Omega Bio-Tek). 250 ng of each sample were denatured in Glyoxal/DMSO RNA loading buffer (Ambion) containing SYBR nucleic acid stain (Molecular Probes). 1μg of each RNA sample was reverse transcribed with RevertAid RNaseH- M-MLV reverse transcriptase 200 u (Fermentas) in 25 μl reactions consisting in addition to the enzymes, own1× buffer, 1 mM dNTPs, 2 μM random nonamer primers (Sigma-Aldrich), 1.5 μl D(+) trehalose (saturated at room temperature), 800 mM D(+) sorbitol, 10 u SUPERase-in RNase inhibitor (Ambion). Samples were incubated 25° C., 5 min; 37° C., 5 min; 42° C., 5 min; 55° C., 5 min; 93° C., 3 min.

2 μl of each RT-reactions was used as template in 20 μl PCR reactions using Dynazyme II polymerase 1 u (Finnzymes) in it's own 1× buffer 100 μM dNTPs (˜the same amount comes with the template from RT-reactions) 2% DMSO, GUS-5′-F and 250 nM GUS-e2-R primers. Program: 95° C. 4 min., 35×[(95° C., 30 s), (52° C., 20 s), (72° C., 30 s)].

The primers for RT-PCR were designed to match the 103 nucleotide sequence (SEQ ID NO:14) from transcription terminator region of ALS gene of Papaver rhoeas (which was introduced close downstream of stop codon the mutated Arabidopsis ALS gene specifically for this purpose). Most of the RNA samples produced the right size amplification product confirming the right mRNA expression of foreign ALS gene.

Southern Analysis

Total genomic DNA was isolated from leaf tissue of Camelina sativa plants by CTAB extraction and DEAE sepharose purification. Leaf material of 10 transgenic ALS plant lines of cv. Blanc Creek of third generation was joined and used for DNA isolation. Southern Analysis was conducted of the samples of transgenic ALS plant lines of third generation:

ALS1.2 (VK12A8.18)

ALS2.2 (KK21A14.1.2)

ALS3.3 (KK21A14.3)

ALS5.1 (VK13A14.1)

ALS7.3 (KK24A14.4)

ALS8.3 (KK18A8/KK20A7)

ALS9.1 (KK18A8/KK20A7)

ALS10.3 (VK12A8.9.1)

ALS11.3 (VK10A5.3)

ALS22.3 (VK13A15.12)

Eight micrograms of total DNA of Camelina sativa plants was digested with XbaI restriction enzyme. These enzyme cut out 5.8 kb fragment of almost whole T-DNA region. Digested DNA samples were separated in a 0.8% agarose gel in overnight electrophoresis at 9 V and 15 mA current and transferred to positively charged nylon membrane (Boehringer, Mannheim) using capillary transfer. RNA probes were synthesized using T7 RNA polymerase on the PCR product carrying promoter of Arabidopsis ALS gene and labeled with digoxigenin-11-UTP. The membrane was hybridized and developed according to the supplier's instructions (Boehringer, Mannheim, The DIG user's guide for filter hybridization): prehybridized at 50° C. for 2 h and hybridized at 50° C. in a “DIG Easy Hyb” hybridization solution (Boehringer, Mannheim) overnight with a digoxigenin-UTP labeled RNA probe. The concentration of RNA probe was 150 ng/ml. After hybridization the membrane was washed in SSC buffers, blocked and detected by using “Anti-Digoxigenin-AP alkaline phosphatase (Boehringer, Mannheim). Chemiluminescent detection was conducted with CSPD-substrate and the membrane was exposed to X-ray film. Presence of the transgene insertion was proved by comparison to DNA of non-transgenic Camelina sativa plant DNA as negative control (line BC+1 kb M), and to plasmid DNA carrying the gene sequence, cut with restriction enzymes and mixed with non-transgenic plant DNA as positive control (line BC+M) (FIG. 15).

The XbaI cut 5.8 kb fragment contains whole ALS gene including promoter and part of terminator regions. Therefore the right size T-DNA insert should look as one 5.8 kb band in FIG. 15. If one recognition site of the XbaI restriction enzymes absence in the case of defective insert, the size of the detected band should differ 5.8 kb.

As a result of the Southern analysis we can say that all analyzed lines contain right size transgenic insert in the genome (FIG. 15). Plant lines of 1.2; 5.1; 7.3 and 10.3 (columns 1,4,5 and 8 in FIG. 15 respectively) do not contain defective inserts. Lines 2.2; 3.3; 8.3; 9.1; 11.3 and 22.3 (columns 2,3, 6,7,9 and 10 in FIG. 15, respectively) have extra bands caused by additional incomplete insert(s) in the genome.

### EXAMPLE 4

### Herbicide Assays

Seedlings of the developed transgenic lines of Camelina sativa, cultivars Blane Creek and Calena second (T1) and fourth (T3) generations were exposed to various concentration of three herbicides: imidazolinone (IMI), chlorsulfuron (CSU) and metsulfuron (MSU). Resistance of the transgenic lines was compared to control non-transgenic seedlings of C. sativa cv. Blane Creek.

Seeds were sterilized in 2% Na-hypocloride and placed on wet filter paper in sterile conditions to allow germination. On the next day well-germinated seedlings were transferred on Petri dishes containing MS (Murasige-Scoog) 0.7% agar (without sucrose or hormones). Each dish contained one from the three herbicides in particular concentration (0.0001, 0.001, 0.01, 0.1, 1.0, 10, 100 mg/l). After one week of cultivation in vitro (+25° C./+18° C.; light/dark) the seedlings were photographed (as shown on the FIGS. 16A-16F). The highest survival concentration was found for each transgenic line and for each herbicide and compared to the non-transgenic control. The results are combined in the Table 3.

The results of the bioassay to herbicide resistance showed that:
1. Almost all transgenic lines bearing mutated ALS gene demonstrated increased herbicide resistance.
2. In the best lines resistance increased in 10 000 times as compared to non-transgenic control.
3. Each mutation has own spectrum of resistance. A—specifically increased resistance to IMI, P—to CSU, and W has a good resistance to IMI and CSU and it was less effective to MSU.
4. The best resistance reveals lines bearing double mutations PW: 1 000-10 000 increase in resistance to CSU and IMI, and 1 000 increase resistance to MSU.
5. Triple mutation (APW) plants also exhibit a good resistance to all three herbicides.

TABLE 3
Highest survival concentrations of herbicides for various transgenic lines of C. sativa.

| | | | | | Highest | |
|---|---|---|---|---|---|---|
| | | | | | surviving | Effect as |
| | | | | | concentration | compared |
| Mutation | Cultivar | Line | Generation | Herbicide | mg/l | to control |
| No | Blane Creek | Wild type | — | IMI | 0.01 | 1 |
| No | Blane Creek | Wild type | — | CSU | 0.001 | 1 |
| No | Blane Creek | Wild type | — | MSU | 0.001 | 1 |
| A122T | Calena | A3 (VK9A3.4.1) | T1 | IMI | 10 | 1 000 |
| | Blane Creek | A9.1 (VK14A9.1) | T1 | | 1.0 | 100 |
| | Blane Creek | A9.2 (VK14A9.4.2) | T1 | | 10 | 1 000 |
| A122T | Calena | A3 (VK9A3.4.1) | T1 | CSU | 0.01 | 10 |
| | Blane Creek | A9.1 (VK14A9.1) | T1 | | 0.001 | 1 |
| | Blane Creek | A9.2 (VK14A9.4.2) | T1 | | 0.01 | 10 |
| A122T | Calena | A3 (VK9A3.4.1) | T1 | MSU | 0.01 | 10 |
| | Blane Creek | A9.1 (VK14A9.1) | T1 | | 0.001 | 1 |
| | Blane Creek | A9.2 (VK14A9.4.2) | T1 | | 0.001 | 1 |
| P197S | Calena | A4 (VK9A4.4.2) | T1 | IMI | 0.1 | 10 |
| | Blane Creek | A10 (VK14A10.16) | T1 | | 0.1 | 10 |
| P197S | Calena | A4 (VK9A4.4.2) | T1 | CSU | 10 | 10 000 |
| | Blane Creek | A10 (VK14A10.16) | T1 | | 1.0 | 1 000 |
| P197S | Calena | A4 (VK9A4.4.2) | T1 | MSU | 0.01 | 10 |
| | Blane Creek | A10 (VK14A10.16) | T1 | | 0.01 | 10 |
| W574L | Blane Creek | A2 (VK10A2.2) | T1 | IMI | 100 | 10 000 |
| | Blane Creek | ALS23.3 | T3 | | 10 | 1 000 |
| | | (VK10A2.3) | | | | |
| W574L | Blane Creek | A2 (VK10A2.2) | T1 | CSU | 10 | 10 000 |
| | Blane Creek | ALS23.3 | T3 | | 1.0 | 1 000 |
| | | (VK10A2.3) | | | | |
| W574L | Blane Creek | A2 (VK10A2.2) | T1 | MSU | 0.1 | 100 |
| | Blane Creek | ALS23.3 | T3 | | 0.1 | 100 |
| | | (VK10A2.3) | | | | |
| PW | Blane Creek | ALS7.3 | T3 | IMI | 10 | 1 000 |
| | | (KK24A14.4) | | | | |
| | Blane Creek | ALS8.3 (KK18A8) | T3 | | 10 | 1 000 |
| P197S- | Blane Creek | ALS7.3 | T3 | CSU | 10 | 10 000 |
| W574L | | (KK24A14.4) | | | | |
| | Blane Creek | ALS8.3 (KK18A8) | T3 | | 10 | 10 000 |
| P197S- | Blane Creek | ALS7.3 | T3 | MSU | 1.0 | 1 000 |
| W574L | | (KK24A14.4) | | | | |
| | Blane Creek | ALS8.3 (KK18A8) | T3 | | 1.0 | 1 000 |
| A122T- | Blane Creek | ALS 11.3 | T3 | IMI | 10 | 1 000 |
| P197S- | | (VK10A5.3) | | | | |
| W574L | Blane Creek | ALS22.3(VK13A1 | T3 | | 10 | 1 000 |
| | | 5.12 | | | | |
| A122T- | Blane Creek | ALS 11.3 | T3 | CSU | 10 | 10 000 |
| P197S- | | (VK10A5.3) | | | | |
| W574L | Blane Creek | ALS22.3(VK13A1 | T3 | | 10 | 10 000 |
| | | 5.12 | | | | |
| A122T- | Blane Creek | ALS 11.3 | T3 | MSU | 0.1 | 100 |
| P197S- | | (VK10A5.3) | | | | |
| W574L | Blane Creek | ALS22.3(VK13A1 | T3 | | 1.0 | 1 000 |
| | | 5.12 | | | | |

### EXAMPLE 5

### Herbicide Selection

Two transformation series were performed to estimate effect of herbicide selection during in vitro regeneration of shoots C. sativa cv. Blane Creek.

Part of explants was cultured without selection. Another part of explants was grown for 5 days without selection and then 3 days 0.01 mg/l IMI. After the 3 days exposure to herbicide the cultivation of explants continued without selection. All recovered shoots were transferred to the MS agar supplied with 0.1 mg/l IMI to ensure the herbicide resistance of the shoots. After 2 -3 weeks of selection, survived shoots were transferred to rooting.

In Table 4 below, the proportion of transgenic shoots (that survived on the second strong selection) is shown in comparison with a number of inoculated explants and all shoots firstly cut from the explants. We did not take into account vitrificated or weakly survived shoots. Thus the real number of transgenic shoots could be much greater than shown in the table. Therefore it is suggested here, that nucleotide sequences encoding mutated ALS-proteins are beneficial to use for purpose of selection and more specifically for purpose of improving frequency of transgenic plants.

TABLE 4
Transformation frequencies in two transformation series VK12 and VK13.
VK12 transformation series: 17^{th} Mar. 2009, Blane Creek, constructs: A2 (pCambia0300 Pals-
ALS(W574)-Tals), A5 (pCambia0300 Pals-ALS (A122, P197, W574)-Tals) and A8
(pCambia0300 Pals-ALS(P197, W574)-Tals)
VK13 transformation series: 26^{th} Mar. 2009, Blane Creek, constructs: A11 (pCambia0300
Pals(-TATA)-ALS(W574)-Tals), A13 (pCambia0300 Pals(-TATA)-ALS(A122 + P197)-Tals),
A14 (pCambia0300 Pals(-TATA)-ALS(P197, W574)-Tals), A15 (pCambia0300 Pals(-TATA)-
ALS(A122, P197, W574)-Tals)

| | Construct | | | All | | Frequency | Frequency |
|---|---|---|---|---|---|---|---|
| | code | IMI | | shoots | Resistant | % to | % to all |
| Transformation | (mutation) | selection | Explants # | cut # | shoots # | explants | shoots |
| VK12A2 | A2 (W574) | NO | 26 | 40 | 12 | 46 | 30 |
| VK12A2 | A5 (A + P + W) | NO | 21 | 25 | 4 | 19 | 16 |
| VK12A2 | A8 (P + W) | NO | 25 | 42 | 8 | 32 | 19 |
| VK13A11 | A11 (W574) | NO | 23 | 29 | 5 | 21 | 17 |
| VK13A13 | A13 (A + P) | NO | 21 | 18 | 0 | 0 | 0 |
| VK13A14 | A14 (P + W) | NO | 19 | 26 | 5 | 26 | 19 |
| VK13A15 | A15 | NO | 9 | 29 | 0 | 0 | 0 |
| | (A + P + W) | | | | | | |
| Total | | | 144 | 209 | 34 | 23.6% | 16.3% |
| Max | | | | | | 46% | 30% |
| Min | | | | | | 0% | 0% |
| VK12A2 | A2 (W574) | YES | 29 | 33 | 14 | 48 | 42 |
| VK12A2 | A5 (A + P + W) | YES | 22 | 24 | 23 | 104 | 96 |
| VK12A2 | A8 (P + W) | YES | 23 | 31 | 9 | 39 | 29 |
| VK13A11 | A11 (W574) | YES | 27 | 28 | 7 | 26 | 25 |
| VK13A13 | A13 (A + P) | YES | 30 | 53 | 20 | 67 | 38 |
| VK13A14 | A14 (P + W) | YES | 26 | 40 | 7 | 27 | 18 |
| VK13A15 | A15 | YES | 26 | 39 | 15 | 58 | 39 |
| | (A + P + W) | | | | | | |
| Total | | | 183 | 248 | 95 | 51.9% | 38.3% |
| Max | | | | | | 104% | 96% |
| Min | | | | | | 26% | 18% |

What is claimed is:

1. A transgenic Camelina sativa plant, said plant being resistant to acetolactate synthase inhibiting herbicides.

2. The plant of claim 1, wherein resistance to acetolactate synthase inhibiting herbicides is achieved by transforming the plant with a construct comprising an ALS natural gene cassette of Arabidopsis thaliana, said gene cassette comprising an ALS promoter, a mutated ALS gene coding region and a terminator region.

3. The plant of claim 2, wherein the mutated coding region comprises one to three point mutations.

4. The plant of claim 3, wherein the mutated ALS gene coding region is according to SEQ ID NO: 24 including one or more point mutations selected from the group consisting of Al22T, P197S and W574L.

5. The plant according to claim 2, 3 or 4 wherein the promoter is according to SEQ ID NO: 9.

6. The plant according to claim 5, wherein TATA-boxes of SEC61 encoding gene are removed by mutation of the promoter region between SacI-PvuI restriction sites.

7. The plant according to claim 6, wherein the promoter is according to SEQ ID NO: 33.

8. The plant according to anyone of claim 2, wherein the terminator is according to SEQ ID NO: 10.

9. A transformation cassette for plant transformation to produce transgenic plant resistant to ALS-binding herbicides, said cassette comprising a nucleotide sequence encoding mutated ALS-protein, said mutated ALS gene coding region further being according to SEQ ID NO: 24 and including one or more point mutations selected from the group consisting of A122T, P197S and W574L.

10. The transformation cassette according to claim 9, wherein the ALS-encoding nucleotide sequence is operably linked to an ALS promoter according to SEQ ID NO: 9.

11. The transformation cassette according to claim 10, wherein TATA-boxes of SEC61 encoding gene are removed by mutation of the promoter region between SacI-PvuI restriction sites.

12. The transformation cassette according to claim 9, wherein ALS-encoding nucleotide sequence is operably linked to an ALS promoter according to SEQ ID NO: 33.

13. A method to improve transformation frequency in in vitro selection, said method comprising:
a) transforming a plant tissue with a construct carrying a nucleotide sequence encoding mutated ALS-protein, and a nucleotide sequence encoding a gene of interest;
b) cultivating the transformed plant tissue in vitro on a medium containing ALS-inhibiting herbicide; and
c) growing and rooting recovered shoots.

14. The method of claim 14, wherein the mutated ALS-protein is encoded by a nucleotide sequence according to SEQ ID NO: 24 including one or more point mutations selected from the group consisting of Al22T, P197S and W574L.
